(19) United States
(12) Patent Application Publication
(10) Pub. No.: US 2020/0224260 A1
(43) Pub. Date: Jul. 16, 2020

(54) METHOD FOR SUPPRESSING NON-SPECIFIC AMPLIFICATION PRODUCTS IN NUCLEIC ACID AMPLIFICATION TECHNOLOGIES

(71) Applicant: Tangen Bioscience Inc., Branford, CT (US)

(72) Inventors: John F. Davidson, Guilford, CT (US); Zheng Xue, Madison, CT (US)

(73) Assignee: Tangen Bioscience Inc., Branford, CT (US)

(21) Appl. No.: 16/688,531

(22) Filed: Nov. 19, 2019

Related U.S. Application Data
(63) Continuation of application No. 62/792,613, filed on Jan. 15, 2019.

(51) Int. Cl. C12Q 1/6853

(52) U.S. Cl. CPC C12Q 1/6853

(57) ABSTRACT

The use of Nucleic Acid Amplification Technologies (NAATs) to rapidly copy a specific fragment of DNA from a few starting molecules has been used to determine the presence of that DNA in a sample. It is of importance for various applications including the identification of a pathogen in a clinical sample. Non-specific DNA amplification occurring in the absence of any input DNA template is frequently observed after many amplification cycles or in the case of isothermal amplification, with time. The disclosed embodiments describe the surprising finding that the amplification of primer-only artefacts is suppressed in reactions where a fraction of the oligos involved in the reaction contain 3′ blocked terminal bases that cannot support extension by DNA polymerases. The effect of these 3′ blocked oligos is to disproportionality retard the primer-only reactions compared to targeted templated primed true positive reactions, thus opening up the window separating false positives from true positives and vastly improving the reaction specificity and sensitivity.

### RELATED APPLICATIONS

This application takes priority to a U.S. Provisional Application USSN 62/792,613, filed Jan. 15, 2019, by John Davidson, entitled “A Method For Suppressing Non-Specific Amplification Products In Nucleic Acid Amplification Technologies”.

### FIELD

This invention relates generally to nucleic acid amplification, and more particularly to methods, compositions, systems and technologies for amplification of nucleic acids that suppress or reduce non-specific amplification products.

### SEQUENCE LISTINGS

The instant application contains a Sequence Listing that has been submitted electronically in ASCII format and is hereby incorporated by reference in its entirety. Said ASCII copy, created on Oct. 31, 2019, is named TNG-0600-UTL_SL.txt and is 14,984 bytes in size.

### BACKGROUND

The following includes information that may be useful in understanding the present inventions. It is not an admission that any of the information provided herein is prior art, or relevant, to the presently described or claimed inventions, or that any publication or document that is specifically or implicitly referenced is prior art.

Nucleic acid analysis methods based on the complementarity of nucleic acid nucleotide sequences can analyze genetic traits directly. Thus, these methods are a very powerful means for identification of genetic diseases, cancer, microorganisms etc. Nevertheless, the detection of a target gene or nucleic acid present in a very small amount in a sample is not easy, and therefore, amplification of the target gene or its detection signal is necessary. As such, in vitro nucleic acid amplification technologies (NAATs) are an invaluable and powerful tool for detection and analysis of small amounts of nucleic acid in many areas of research and diagnosis.

NAAT techniques allow detection and quantification of a nucleic acid in a sample with high sensitivity and specificity. NAAT techniques may be used to determine the presence of a particular template nucleic acid in a sample, as indicated by the presence of an amplification product (i.e., amplicon) following the implementation of a particular NAAT. Conversely, the absence of any amplification product indicates the absence of template nucleic acid in the sample. Such techniques are of great importance in diagnostic applications, for example, for determining whether a pathogen is present in a sample. Thus, NAAT techniques are useful for detection and quantification of specific nucleic acids for diagnosis of infectious and genetic diseases.

NAATs can be grouped according to the temperature requirements of the procedure. For example, the polymerase chain reaction (PCR) is the most popular method as a technique of amplifying nucleic acid in vitro. This method was established firmly as an excellent detection method by virtue of high sensitivity based on the effect of exponential amplification. Further, since the amplification product can be recovered as DNA, this method is applied widely as an important tool supporting genetic engineering techniques such as gene cloning and structural determination. In the PCR method, however, temperature cycling or a special temperature controller is necessary for practice; the exponential progress of the amplification reaction causes a problem in quantification; and samples and reaction solutions are easily contaminated from the outside to permit nucleic acid mixed in error to function as a template (See R. K. Saiki, et al. 1985. Science 230, 1350-1354). Other PCR-based amplification techniques, for example, transcription-based amplification (D. Y. Kwoh, et at. 1989. Proc. Natl. Acad Sci. USA 86, 1173-1177), ligase chain reaction (LCR; D. Y. Wu, et al. 1989. Genomics 4, 560-569; K. Barringer, et al. 1990. Gene 89, 117-122; F. Barany. 1991. Proc. Natl. Acad. Sci. USA 88, 189-193), and restriction amplification (U.S. Pat. No. 5,102,784) similarly require temperature cycling.

Many NAATs use strand displacement polymerase (SD pols) to enable isothermal amplification of target template DNA or RNA molecules. A common feature of SD pol is a higher affinity of the polymerase for primer:template complexes resulting in higher processivity and strand displacement. Strand displacement is utilized in a variety of isothermal amplification strategies such as Loop Mediated Amplification (LAMP), Strand Displacement Amplification (SDA), Cross Priming Amplification (CPA), Rolling Circle Amplification (RCA) and hyperbranched RCA (HRCA), Recombinase Polymerase Amplification (RPA) and Helicase Dependent Amplification. Many of these NAATs are used to detect pathogen DNA with high sensitivity and specificity for use in diagnostic applications.

While extensively used, LAMP has been observed to be less sensitive than PCR to inhibitors in complex samples such as blood, likely due to use of a different DNA polymerase (typically Bst DNA polymerase rather than Taq polymerase as in PCR). LAMP is useful primarily as a diagnostic or detection technique, but is not useful for cloning or myriad other molecular biology applications enabled by PCR.

A common problem with these and other polymerase based NAATs is that undesirable polymerase-based primer-only reactions can lead to the formation of non-specific amplification products that compete with the target template derived reaction of interest. Efforts have been made during the design of primers to exclude primers that have strongly stabilized interactions with each other to reduce the propensity and delay the appearance of polymerase-based primer derived false positive reactions. Further, software applications that facilitate the process of primer design are used and despite these measures, extensive screening reactions with many different primer sets are often necessary to find one that has sufficiently slow appearance of No Template False Positives (NTFPs) compared to Templated True Positives (TTPs). An inherent problem is that, given enough time, all isothermal NAATs will produce NTFPs. There is still an unmet need for compositions, methods, and systems that reduce undesirable no template false positive (NTFP) amplification products.

The inventions described herein meet these unsolved challenges and needs. As described in detail herein below, novel embodiments of the invention described herein suppress or reduce non-specific amplification products using different approaches. The inventions have other benefits, including significant improvements to the reaction sensitivity and specificity and allowing fewer primer designs to be developed and screened for amplification reactions.

### BRIEF SUMMARY

The inventions described and claimed herein have many attributes and embodiments including, but not limited to, those set forth or described or referenced in this Brief Summary. The inventions described and claimed herein are not limited to, or by, the features or embodiments identified in this Summary, which is included for purposes of illustration only and not restriction.

Aspects of the invention relate to compositions, methods, and systems for detecting or quantifying a target nucleic acid in a nucleic acid sample and reducing the amplification of non-template molecules from the sample.

Thus in one aspect, methods of detecting or quantifying a target nucleic acid in a nucleic acid sample and reducing the amplification of non-template molecules from the sample are described and provided herein. An exemplary embodiment comprises the steps of i) incubating a composition comprising a nucleic acid sample comprising a template; one or more first amplification primer set(s); one or more second primer set(s); a polymerase; and deoxynucleotide triphosphates, ii) amplifying the template, wherein the one or more first primer set(s) and the one or more second primer set(s) compete for binding with the template in step i) and/or step ii), and the inclusion of one or more second primer set(s) in the composition reduces non-specific amplification products in step ii).

Further embodiments of the above exemplary embodiment often comprise the step of quantifying the amount of amplified template. The template nucleic acid sample typically, but not necessarily, comprises a target nucleic acid in these exemplary embodiments. In many variants of the exemplary embodiment described above, the one or more first primer set(s) is greater in length than the one or more second primer set(s). In many variants of the exemplary embodiment described above, the second primer set has one or more mismatched nucleotide with the template. In some embodiments, the one or more second primer set(s) has two mismatched nucleotides with the template. In many variants of the exemplary embodiment described above, the one or more first primer set(s) have a higher binding affinity for the template in the composition than the one or more second primer set(s). In many variants of the exemplary embodiment described above, the one or more second primer set(s) comprises modified or non-natural nucleotide analogs. In many variants of the exemplary embodiment described above, the one or more second primer set(s) has a modified backbone or a modified 3′ terminal nucleotide.

In many variants of the exemplary embodiment described above, in the amplification step ii), the modified 3′ terminal nucleotide reduces the amount of amplification of products comprising the one or more second primer sets relative to the amount of amplification of products comprising the one or more first primer sets. In many variants of the exemplary embodiment described above, the one or more second primer set(s) is between 5% and 200% of the total of first and second primer sets in the composition, between 5% and 50% of the total of first and second primer sets in the composition, between 5% and 40% of the total of first and second primer sets in the composition, between 5% and 30% of the total of first and second primer sets in the composition, between 10% and 100% of the total of first and second primer sets in the composition, between 10% and 70% of the total of first and second primer sets in the composition, between 10% and 50% of the total of first and second primer sets in the composition, between 10% and 40% of the total of first and second primer sets in the composition, between 10% and 30% of the total of first and second primer sets in the composition, between 20% and 70% of the total of first and second primer sets in the composition, between 30% and 60% of the total of first and second primer sets in the composition, or between 40% and 50% of the total of first and second primer sets in the composition. In many variants of the exemplary embodiment described above, the one or more second primer set(s) is between 15 and 25% of the total percentage by weight of first and second primer sets in the composition. In certain embodiments, the one or more second primer set(s) is between 5 and 30% of the total percentage by weight of first and second primer sets in the composition.

In many variants of the exemplary embodiment described above, the reaction mixture is an amplification reaction mixture selected from a loop-mediated (LAMP) reaction mixture, stand displacement reaction mixture (SDS), Polymerase Chain Reaction (PCR), a ligase chain reaction (LCR), Isothermal Chimeric Amplification of Nucleic Acids (ICAN), SMart Amplification Process (SMAP), Chimeric Displacement Reaction (RDC), (exponential)-rolling circle amplification (exponential-RCA), Nucleic Acid Sequence Based Amplification (NASBA), Transcription Mediated Amplification (TMA), and Helicase Dependent Amplification (HAD) and Recombinase polymerase amplification (RPA). In certain embodiments, the polymerase is selected from a strand-displacing polymerase and a thermostable polymerase.

In another aspect of the invention, compositions are provided. An exemplary embodiment of a composition according to the invention comprises: a) a nucleic acid sample comprising a template; b) one or more first amplification primer sets; c) one or more second primer sets; d) a polymerase; and e) deoxynucleotide triphosphates, where the composition is i) capable of amplifying the template when placed under amplification conditions, wherein the one or more first primer set(s) and ii) the one or more second primer set(s) compete for binding with the template, and the inclusion of one or more second primer sets in the composition reduces non-specific amplification products when the template is amplified. In the above embodiments, the composition typically, but not necessarily, comprise a reaction mixture. Typically, but not necessarily, the template nucleic acid sample comprises a target nucleic acid. An exemplary template nucleic acid sample comprises genomic DNA.

In another aspect, apparatus, systems and the like for performing the methods described herein are provided. An exemplary embodiment of an apparatus and system for performing nucleic acid amplification comprises: i) a central chamber for performing an amplification reaction of an amplification composition or reaction mixture, said amplification reaction mixture comprising a) a nucleic acid sample comprising a template; b) one or more first amplification primer set(s); c) one or more second primer set(s); d) a polymerase; and e) deoxynucleotide triphosphates, wherein during an amplification reaction performed in the system, the one or more first primer set(s) and the one or more second primer set(s) compete for binding with the template and the inclusion of one or more second primer set(s) in the composition reduces non-specific amplification products, wherein, optionally, the central chamber is in communication with one or more ii) additional chambers, in which, one or more additional amplification reactions takes place; iii) an instrument for detecting and comparing in real-time the amplification rates of the at least two secondary reactions; and optionally iv) a reaction mixture and reagents for performing a nucleic acid amplification and real-time detection method in the system.

In another aspect, kits for detecting or quantifying a target nucleic acid in a nucleic acid sample are described herein. Such a kit may comprise any of the apparatus, compositions and systems described herein and may be utilized in any method described herein. Accordingly, certain embodiments are directed to kits for performing methods of detecting or quantifying a target nucleic acid in a nucleic acid sample and reducing the amplification of non-template molecules from the sample.

In another aspect, other embodiments described herein are directed to a multiplexed nucleic acid amplification and real-time detection method. An exemplary two-stage embodiment of the method comprises the steps of a) providing a composition comprising a target nucleic acid sample comprising a template having a region of interest, one or more first amplification primer sets, one or more second primer sets, a polymerase, and deoxynucleotide triphosphates; b) performing a first reaction to amplify the region of interest (first-stage reaction), thereby forming a primary amplicon; c) dividing (b) into at least two secondary reactions, and including in at least one of the reactions one or more site-specific secondary primer that is complementary to a site-specific primer binding site that may be present within the primary amplicon and defines a site of interest within the region of interest; and d) performing a second reaction (second-stage reaction) thereby accelerating the amplification of the region of interest only if the site-specific primer binding site is complementary to the site-specific primer; and e) detecting and comparing the amplification rates of the at least two secondary reactions, wherein an enhanced relative rate of amplification in the reaction with the secondary primer indicates the presence of the site of interest that is complementary to the secondary primer.

### BRIEF DESCRIPTION OF THE DRAWINGS

FIG. 1 shows the application of blocked oligos to an isothermal LAMP reaction.

FIG. 2 shows a LAMP reaction targeting Candida albicans. C_alb1 is a LAMP primer set that targets the 18s rRNA gene of Candida albicans. The X axis identifies the distribution of true positives (2000 genomes of C. albicans DNA) and negatives wells on a 96 well qPCR Plate for each condition of tested while the Y axis identifies the Cq (quantification cycle, 1 cycle=15 seconds) or speed of the reaction. The secondary Y axis identifies the % NTP frequency which is the frequency of wells showing NTPs within 300 scans. Varying concentrations of the blocked primer (full length primer with a 3′ phosphate blocking group) combined with the un-blocked original primer set were tested to determine the effect of increasing concentration of un-blocked to blocked primer. MS14 (positive LAMP control, containing 2000 genomes Mycobaterium smegmatis) and Bst2.0 (24 units) was also added to the reaction, serving as a positive control.

FIG. 3 shows a Candida Albicans 8mer blocked Cq distribution. C_alb1 is a LAMP primer set that targets the 18s rRNA gene of Candida albicans. The X axis identifies the distribution of true positives (2000 genomes of C. albicans genomic DNA) and negatives wells on a 96 well qPCR Plate for each condition of tested while the Y axis identifies the Cq (quantification cycle, 1 cycle=15 seconds) or speed of the reaction. The secondary Y axis identifies the % NTP frequency which is the frequency of wells showing NTPs within 300 scans. Varying concentrations of the blocked primer (8mer primer with a Phosphate 3′ blocking group, C_Alb1_8merP) combined with the un-blocked original primer set were tested to determine the optimum concentration of un-blocked to blocked primer.

FIG. 4 illustrates the effect of varying blocked oligonucleotide concentration in Candida albicans. C_Alb1 is a LAMP primer set that targets the 18s rRNA gene of Candida albicans. The X axis identifies the percentage of additional C_Alb_P, 3′ blocked oligos (3′ phosphate) added to a standard LAMP reaction as well as the control with just the active C_Alb primers containing no additional C_Alb_P, 3′ blocked oligos. The Y axis identifies the Cq gap (ΔCq) between the slowest true positive and the fastest no-template false positive. PCR Control (positive LAMP control unaffected by the blocking oligos to C_Alb1, containing 2000 genomes Mycobacterium smegmatis) was also added to the reaction, serving as a positive reaction control.

FIG. 5 illustrates the effect on positive speed and Cq gap between slowest positive and fastest NTP in Candida albicans. C_alb1 is a LAMP primer set that targets the 18s rRNA gene of Candida albicans. The X axis identifies each concentration of C_alb_8merP (blocked), tested as well as the control with no C_alb_8merP (unblocked). The Y axis identifies the Cq (quantification cycle, 1 cycle=15 seconds) or speed of the reaction, the secondary Y axis identifies the Cq gap between the slowest true positive and the fastest no-template positive well.

FIG. 6 shows the effect of blocking full length oligonucleotides in Klebsiella pneumoniae. KPC2f is a LAMP primer set that targets the KPC2 gene of Klebsiella pneumoniae. The X axis identifies the distribution of true positives (2000 genomes Klebsiella pneumoniae (KPC2+)) and negatives wells on a 96 well qPCR Plate for each condition of tested while the Y axis identifies the Cq (quantification cycle, 1 cycle=15 seconds) or speed of the reaction. The secondary Y axis identifies the % NTP frequency which is the frequency of wells showing NTPs within 300 scans. Varying concentrations of the blocked primer (full length primer with a 3′ Phosphate blocking group, KPC2f_P) combined with the un-blocked original primer set were tested to determine the optimum concentration of un-blocked to blocked primer.

FIG. 7 shows the effect of blocking 8mer oligonucleotides in Klebsiella pneumoniae. KPC2f is a LAMP primer set that targets the KPC2 gene of Klebsiella pneumoniae. The X axis identifies the distribution of positives and negatives wells on a 96 well qPCR Plate for each condition of tested while the Y axis identifies the Cq (quantification cycle, 1 cycle=15 seconds) or speed of the reaction. The secondary Y axis identifies the % NTP frequency which is the frequency of wells showing NTPs within 300 scans. Varying concentrations of the blocked primer (8mer primer with a 3′Phosphate blocking group, KPC2f_8merP) combined with the un-blocked original primer set were tested to determine the optimum concentration of un-blocked to blocked primer.

FIG. 8 shows the effect of varying the blocking oligonucleotide length. KPC2f is a LAMP primer set that targets the KPC2 gene of Klebsiella and Ecoli. The X axis identifies the Cq of KPC2 Positives true positive LAMP reactions containing 2000 genomes of Klebsiella pseudomonas (KPC2+) DNA as well as NTP Cq and frequency for No Template False Positive wells (NTP) on a 96 well qPCR Plate for each condition. The left Y axis identifies the Cq (quantification cycle, 1 cycle=15 seconds) or speed of the reaction. Each dot represents a single well Cq. The frequency of false positives (No template positives, NTP) is represented with an x symbol and the right Y axis designates % frequency. KPC2f oligos with a 3′ Phosphate blocking group and oligo lengths varying from 8nt (KPC2_8merP), 10 nt (KPC2_10merP), and 12 nt (KPC2_12merP), were added at 50% the standard LAMP oligo concentration along with 100% standard unblocked oligo where indicated.

FIG. 9 shows the effect of varying the 3′ blocking group chemistry of the oligonucleotide. C_fs1 is a LAMP primer set that targets the KPC2 gene of Klebsiella and Ecoli. The X axis identifies the Cq of KPC2 Positives true positive LAMP reactions containing 2000 genomes of Klebsiella pseudomonas (KPC2+) as well as NTP Cq and frequency for No Template False Positive wells (NTP) on a 96 well qPCR Plate for each condition. The left Y axis identifies the Cq (quantification cycle, 1 cycle=15 seconds) or speed of the reaction. Each dot represents a single well Cq. The frequency of false positives (No template positives, NTP) is represented with an x symbol and the right Y axis designates % frequency. Full length oligos with either a 3′ Phosphate blocking group (KPC2_P), a 3′ 3 carbon (KPC2_3C3) or an additional 3′ dideoxy C base blocking group (KPC_ddp) were added at 50% the standard LAMP oligo concentration along with 100% standard unblocked oligo where indicated.

FIG. 10 shows the effect of blocking oligonucleotides in LAMP reactions targeting the vanA gene. VanA5 is a LAMP primer set that targets the vanA gene of Enterococcus faecium (vanA+). The X axis identifies the distribution of positives and negatives wells on a 96 well qPCR Plate for each condition of tested while the Y axis identifies the Cq (quantification cycle, 1 cycle=15 seconds) or speed of the reaction. The secondary Y axis identifies the % NTP frequency which is the frequency of wells showing NTPs within 300 scans. Varying concentrations of the blocked primer (full length primer with a Phosphate blocking group, VanA5_P) combined with the un-blocked original primer set were tested to determine the optimum concentration of un-blocked to blocked primer. Van5_P oligos with a 3′ phosphate blocking group were added at 50% the standard LAMP oligo concentration along with 100% standard unblocked Van5_P primers (Van5_P 50%).

FIG. 11 shows the effect of blocking on a 8mer oligonucleotide in the vanA gene. VanA5 is a LAMP primer set that targets the vanA gene of Enterococcus faecium (vanA+). The X axis identifies the distribution of positives and negatives wells on a 96 well qPCR Plate for each condition of tested while the Y axis identifies the Cq (quantification cycle, 1 cycle=15 seconds) or speed of the reaction. The secondary Y axis identifies the % NTP frequency which is the frequency of wells showing NTPs within 300 scans. Varying concentrations of the blocked primer (8mer primer with a Phosphate blocking group, VanA5_8merP) combined with the un-blocked original primer set were tested to determine the optimum concentration of un-blocked to blocked primer.

### DETAILED DESCRIPTION

Various aspects of the invention will now be described with reference to the following section which will be understood to be provided by way of illustration only and not to constitute a limitation on the scope of the invention.

“Complementarity” refers to the ability of a nucleic acid to form hydrogen bond(s) or hybridize with another nucleic acid sequence by either traditional Watson-Crick or other non-traditional types. As used herein “hybridization,” refers to the binding, duplexing, or hybridizing of a molecule only to a particular nucleotide sequence under low, medium, or highly stringent conditions, including when that sequence is present in a complex mixture (e.g., total cellular) DNA or RNA. See e.g. Ausubel, et al., Current Protocols In Molecular Biology, John Wiley & Sons, New York, N.Y., 1993. If a nucleotide at a certain position of a polynucleotide is capable of forming a Watson-Crick pairing with a nucleotide at the same position in an anti-parallel DNA or RNA strand, then the polynucleotide and the DNA or RNA molecule are complementary to each other at that position. The polynucleotide and the DNA or RNA molecule are “substantially complementary” to each other when a sufficient number of corresponding positions in each molecule are occupied by nucleotides that can hybridize or anneal with each other in order to affect the desired process. A complementary sequence is a sequence capable of annealing under stringent conditions to provide a 3′-terminal serving as the origin of synthesis of complementary chain.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” and “similarity” can be readily calculated by known methods, including, but not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., Siam J. Applied Math., 48:1073 (1988). In addition, values for percentage identity can be obtained from amino acid and nucleotide sequence alignments generated using the default settings for the AlignX component of Vector NTI Suite 8.0 (Informax, Frederick, Md.). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215:403-410 (1990)). The BLAST X program is publicly available from NCBI and other sources (BLAST Manual, Altschul, S., et al., NCBINLM NIH Bethesda, Md. 20894: Altschul, S., et al., J. Mol. Biol. 215:403-410 (1990). The well-known Smith Waterman algorithm may also be used to determine identity.

The terms “amplify”, “amplifying”, “amplification reaction”, or a “NAAT” and their variants, refer generally to any action or process whereby at least a portion of a nucleic acid molecule (referred to as a template nucleic acid molecule) is replicated or copied into at least one additional nucleic acid molecule. The additional nucleic acid molecule optionally includes sequence that is substantially identical or substantially complementary to at least some portion of the template nucleic acid molecule. The template nucleic acid molecule can be single-stranded or double-stranded and the additional nucleic acid molecule can independently be single-stranded or double-stranded. In some embodiments, amplification includes a template-dependent in vitro enzyme-catalyzed reaction for the production of at least one copy of at least some portion of the nucleic acid molecule or the production of at least one copy of a nucleic acid sequence that is complementary to at least some portion of the nucleic acid molecule. Amplification optionally includes linear or exponential replication of a nucleic acid molecule. In some embodiments, such amplification is performed using isothermal conditions; in other embodiments, such amplification can include thermocycling. In some embodiments, the amplification is a multiplex amplification that includes the simultaneous amplification of a plurality of target sequences in a single amplification reaction. At least some of the target sequences can be situated, on the same nucleic acid molecule or on different target nucleic acid molecules included in the single amplification reaction. In some embodiments, “amplification” includes amplification of at least some portion of DNA- and RNA-based nucleic acids alone, or in combination. The amplification reaction can include single or double-stranded nucleic acid substrates and can further including any of the amplification processes known to one of ordinary skill in the art. In some embodiments, the amplification reaction includes polymerase chain reaction (PCR). In the present invention, the terms “synthesis” and “amplification” of nucleic acid are used. The synthesis of nucleic acid in the present invention means the elongation or extension of nucleic acid from an oligonucleotide serving as the origin of synthesis. If not only this synthesis but also the formation of other nucleic acid and the elongation or extension reaction of this formed nucleic acid occur continuously, a series of these reactions is comprehensively called amplification.

The terms “target primer” or “target-specific primer” and variations thereof refer to primers that are complementary to a binding site sequence. Target primers are generally a single stranded or double-stranded polynucleotide, typically an oligonucleotide, that includes at least one sequence that is at least partially complementary to a target nucleic acid sequence.

“Forward primer binding site” and “reverse primer binding site” refers to the regions on the template DNA and/or the amplicon to which the forward and reverse primers bind. The primers act to delimit the region of the original template polynucleotide which is exponentially amplified during amplification. In some embodiments, additional primers may bind to the region 5′ of the forward primer and/or reverse primers. Where such additional primers are used, the forward primer binding site and/or the reverse primer binding site may encompass the binding regions of these additional primers as well as the binding regions of the primers themselves. For example, in some embodiments, the method may use one or more additional primers which bind to a region that lies 5′ of the forward and/or reverse primer binding region. Such a method was disclosed, for example, in WO0028082 which discloses the use of “displacement primers” or “outer primers”.

In some embodiments, amplification can be performed using multiple target-specific primer pairs in a single amplification reaction, wherein each primer pair includes a forward target-specific primer and a reverse target-specific primer, each including at least one sequence that substantially complementary or substantially identical to a corresponding target sequence in the sample, and each primer pair having a different corresponding target sequence. In some embodiments, the target-specific primer can be substantially non-complementary at its 3′ end or its 5′ end to any other target-specific primer present in an amplification reaction. In some embodiments, the target-specific primer can include minimal cross hybridization to other target-specific primers in the amplification reaction. In some embodiments, target-specific primers include minimal cross-hybridization to non-specific sequences in the amplification reaction mixture. In some embodiments, the target-specific primers include minimal self-complementarity. In some embodiments, the target-specific primers can include one or more cleavable groups located at the 3′ end. In some embodiments, the target-specific primers can include one or more cleavable groups located near or about a central nucleotide of the target-specific primer. In some embodiments, one of more targets-specific primers includes only non-cleavable nucleotides at the 5′ end of the target-specific primer. In some embodiments, a target specific primer includes minimal nucleotide sequence overlap at the 3′end or the 5′ end of the primer as compared to one or more different target-specific primers, optionally in the same amplification reaction. In some embodiments 1, 2, 3, 4, 5, 6, 7, 8, 9, 10 or more, target-specific primers in a single reaction mixture include one or more of the above embodiments. In some embodiments, substantially all of the plurality of target-specific primers in a single reaction mixture includes one or more of the above embodiments.

The terms “identity” and “identical” and their variants, as used herein, when used in reference to two or more nucleic acid sequences, refer to similarity in sequence of the two or more sequences (e.g., nucleotide or polypeptide sequences). In the context of two or more homologous sequences, the percent identity or homology of the sequences or subsequences thereof indicates the percentage of all monomeric units (e.g., nucleotides or amino acids) that are the same (i.e., about 70% identity, preferably 75%, 80%, 85%, 90%, 95%, 97%, 98% or 99% identity). The percent identity can be over a specified region, when compared and aligned for maximum correspondence over a comparison window, or designated region as measured using a BLAST or BLAST 2.0 sequence comparison algorithms with default parameters described below, or by manual alignment and visual inspection. Sequences are said to be “substantially identical” when there is at least 85% identity at the amino acid level or at the nucleotide level. Preferably, the identity exists over a region that is at least about 25, 50, or 100 residues in length, or across the entire length of at least one compared sequence. A typical algorithm for determining percent sequence identity and sequence similarity are the BLAST and BLAST 2.0 algorithms, which are described in Altschul et al, Nuc. Acids Res. 25:3389-3402 (1977). Other methods include the algorithms of Smith & Waterman, Adv. Appl. Math. 2:482 (1981), and Needleman & Wunsch, J. Mol. Biol. 48:443 (1970), etc. Another indication that two nucleic acid sequences are substantially identical is that the two molecules or their complements hybridize to each other under stringent hybridization conditions.

The polynucleic acid produced by the amplification technology employed is generically referred to as an “amplicon” or “amplification product.” The nature of amplicon produced varies significantly depending on the NAAT being practiced. For example, NAATs such as PCR may produce amplicon which is substantially of identical size and sequence. Other NAATs produce amplicon of very varied size wherein the amplicon is composed of different numbers of repeated sequences such that the amplicon is a collection of concatamers of different length. The repeating sequence from such concatamers will reflect the sequence of the polynucleic acid which is the subject of the assay being performed. In the present specification, the simple expression “5′-side” or “3′-side” refers to that of a nucleic acid chain serving as a template, wherein the 5′ end generally includes a phosphate group and a 3′ end generally includes a free —OH group.

In one aspect, the inventions provided herein relate to increasing the performance and/or specificity of NAAT's by reducing the occurrence of non-specific amplification products in NATT's, such as, for example, conventional amplification techniques such as isothermal amplification techniques.

In an exemplary embodiment, a method of detecting or quantifying a target nucleic acid in a nucleic acid sample and reducing the amplification of non-template molecules from the sample comprises i) incubating a composition comprising a nucleic acid sample comprising the following: a template; one or more first amplification primer set(s); one or more second primer set(s); a polymerase; and deoxynucleotide triphosphates; ii) amplifying the template; wherein the one or more first primer set(s) and the one or more second primer set(s) compete for binding with the template, and the inclusion of one or more second primer set(s) in the composition reduces non-specific amplification products; and iii) quantifying the amount of amplified template.

In some preferred embodiments, an amplification reaction in step ii) above is isothermal. In some embodiments, single-stage isothermal amplification methods are provided that have an increased performance, an increased specificity, and/or a reduced production of non-specific amplification products. The particular type of isothermal reaction used in certain of these embodiments may be any isothermal NAAT (iNAAT) described herein. In other embodiments, single-stage non-isothermal NAAT's (e.g. PCR) are provided that have an increased performance, an increased specificity, and/or a reduced production of non-specific amplification products. The individual cycles of amplification in a non-isothermal amplification reaction such as PCR are not considered ‘stages’ of an amplification and thus non-isothermal multi cycle amplification reactions are not considered multi-stage amplifications herein.

In some embodiments, more than one amplification is performed and the separate amplifications are referenced herein as stages or stages of amplification. Unless explicitly expressed otherwise, any of the amplification techniques or NAAT's described herein can be used in combination in some embodiments of the methods of increasing the performance and specificity of amplification reactions described herein. Thus, an isothermal type amplification reaction such as LAMP can be combined with a non-isothermal amplification such as PCR, or as another example, another isothermal amplification such as a Helicase Dependent Amplification (HAD) reaction. The amplification that is performed first sequentially is the first-stage amplification reaction, the amplification that is performed second sequentially is termed the second-stage amplification reaction, the amplification that is performed third sequentially is termed the third-stage amplification reaction, and so on. The inventors envision that any combination of NAAT's can be used in two-stage, three-stage, four-stage, or other multi-stage amplification embodiments of the invention described and provided herein.

A number of isothermal amplification techniques (iNAATs) can be utilized in embodiments of the invention. Many of these approaches are mentioned above, and some in particular will be described in greater detail. Isothermal amplification techniques typically utilize DNA polymerases with strand-displacement activity, thus eliminating the high temperature melt cycle that is required for PCR. This allows isothermal techniques to be faster and more energy efficient than PCR, and also allows for simpler and lower cost instrumentation since rapid temperature cycling is not required. For example, some methods of the instant invention are directed toward the improvement of conventional iNAAT's such as Strand Displacement Amplification (SDA; G. T. Walker, et at. 1992. Proc. Natl. Acad. Sci. USA 89, 392-396; G. T. Walker, et al. 1992. Nuc. Acids. Res. 20, 1691-1696; U.S. Pat. No. 5,648,211 and EP 0 497 272, all disclosures being incorporated herein by reference); self-sustained sequence replication (3SR; J. C. Guatelli, et al. 1990. Proc. Natl. Acad. Sci. USA 87, 1874-1878, which is incorporated herein by reference); and Q.beta. replicase system (P. M. Lizardi, et al. 1988. BioTechnology 6, 1197-1202, which is incorporated herein by reference) are isothermal reactions (See also, Nucleic Acid Isothermal Amplification Technologies—A Review. Nucleosides, Nucleotides and Nucleic Acids, 2008. v27(3):224-243, which is incorporated herein by reference).

Some isothermal amplification techniques are dependent on transcription as part of the amplification process, for example Nucleic Acid Sequence Based Amplification (NASBA; U.S. Pat. No. 5,409,818) and Transcription Mediated Amplification (TMA; U.S. Pat. No. 5,399,491) while others are dependent on the action of a Helicase or Recombinase for example Helicase Dependent Amplification (HDA; WO2004027025) and Recombinase polymerase amplification (RPA; WO03072805) respectively, others still are dependent on the strand displacement activity of certain DNA polymerases, for example Strand Displacement Amplification (SDA; U.S. Pat. No. 5,455,166), Loop-mediated Isothermal Amplification (LAMP; WO0028082, WO0134790, WO0224902), Chimera Displacement Reaction (RDC; WO9794126), Rolling Circle Amplification (RCA; Lizardi, P. M. et al. Nature Genetics, (1998) 19.225-231), Isothermal Chimeric Amplification of Nucleic Acids (ICAN; WO0216639), SMart Amplification Process (SMAP; WO2005063977), Linear Isothermal Multimerization Amplification (LIMA; Isothermal amplification and multimerization of DNA by Bst DNA polymerase, Hafner G. J., Yang I. C., Wolter L. C., Stafford M. R., Giffard P. M, BioTechniques, 2001, vol. 30, no 4, pp. 852-867) also methods as described in U.S. Pat. No. 6,743,605 (herein referred to as ‘Template Re-priming Amplification’ or TRA) and WO9601327 (herein referred to as ‘Self Extending Amplification’ or SEA).

The methods as described herein can be practiced with any NAAT, including non-isothermal technologies. For example, known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; U.S. Pat. Nos. 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; U.S. Pat. No. 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the entire contents of which references are incorporated herein by reference. (See, e.g., Ausubel, supra; or Sambrook, supra.).

For instance, polymerase chain reaction (PCR) technology can be used to amplify the sequences of polynucleotides of the present invention and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). Additionally, e.g., the T4 gene 32 protein (Boehringer Mannheim) can be used to improve yield of long PCR products.

A common characteristic of the NAATs described herein is that they provide for both copying of a polynucleic acid via the action of a primer or set of primers and for re-copying of said copy by a reverse primer or set of primers. This enables the generation of copies of the original polynucleic acid at an exponential rate. With reference to NAATs in general it is helpful to differentiate between the physical piece of nucleic acid being detected by the method, from the first copy made of this original nucleic acid, from the first copy of the copy made from this original nucleic acid, from further copies of this copy of a copy. A nucleic acid whose origin is from the sample being analyzed itself will be referred to as the “target nucleic acid template.” With reference to the two-stage embodiments described herein, generally, but not always, the first-stage primer-dependent amplification reaction is relatively slow as compared to the second-stage reaction.

As would be understood by the skilled artisan, a primer-generated amplicon gives rise to further generations of amplicons through repeated amplification reactions of the target nucleic acid template as well as priming of the amplicons themselves. It is possible for amplicons to be comprised of combinations with the target template.

The amplicon may be of very variable length as the target template can be copied from the first priming site beyond the region of nucleic acid delineated by the primers employed in a particular NAAT. In general, a key feature of a NAAT in an embodiment herein, whether it is one-step, two-step, or multistep NAAT reaction, will be to provide a method by which the amplicon can be made available to another primer employed by the methodology so as to generate (over repeated amplification reactions) amplicons that will be of a discrete length delineated by the primers used. A key feature of the NAAT is to provide a method by which the amplicons are available for further priming by a reverse primer in order to generate further copies. For some NAATs, the later generation amplicons may be substantially different from the first-generation amplicon, in particular, the formed amplicon may be a concatamer of the first-generation amplicon.

An exemplary target template used in the present invention includes any polynucleic acid that comprises suitable primer binding regions that allow for amplification of a polynucleic acid of interest. The skilled person will understand that the forward and reverse primer binding sites need to be positioned in such a manner on the target template that the forward primer binding region and the reverse primer binding region are positioned 5′ of the sequence which is to be amplified on the sense and antisense strand, respectively. The target template may be single or double stranded. Where the target template is a single stranded polynucleic acid, the skilled person will understand that the target template will initially comprise only one primer binding region. However, the binding of the first primer will result in synthesis of a complementary strand which will then contain the second primer binding region. The target template may be derived from an RNA molecule, in which case the RNA needs to be transcribed into DNA before practicing the method of the invention. Suitable reagents for transcribing the RNA are well known in the art and include, but are not limited to, reverse transcriptase.

The terms “nucleic acid,” “polynucleotides,” and “oligonucleotides” refers to biopolymers of nucleotides and, unless the context indicates otherwise, includes modified and unmodified nucleotides, and both DNA and RNA, and modified nucleic acid backbones. For example, in certain embodiments, the nucleic acid is a peptide nucleic acid (PNA) or a locked nucleic acid (LNA). Typically, the methods as described herein are performed using DNA as the nucleic acid template for amplification. However, nucleic acid whose nucleotide is replaced by an artificial derivative or modified nucleic acid from natural DNA or RNA is also included in the nucleic acid of the present invention insofar as it functions as a template for synthesis of complementary chain. The nucleic acid of the present invention is generally contained in a biological sample. The biological sample includes animal, plant or microbial tissues, cells, cultures and excretions, or extracts therefrom. In certain aspects, the biological sample includes intracellular parasitic genomic DNA or RNA such as virus or mycoplasma. The nucleic acid may be derived from nucleic acid contained in said biological sample. For example, genomic DNA, or cDNA synthesized from mRNA, or nucleic acid amplified on the basis of nucleic acid derived from the biological sample, are preferably used in the described methods. Unless denoted otherwise, whenever a oligonucleotide sequence is represented, it will be understood that the nucleotides are in 5′ to 3′ order from left to right and that “A” denotes deoxyadenosine, “C” denotes deoxycytidine, “G” denotes deoxyguanosine, “T” denotes thymidine, and “U” denotes deoxyuridine. Oligonucleotides are said to have “5′ ends” and “3′ ends” because mononucleotides are typically reacted to form oligonucleotides via attachment of the 5′ phosphate or equivalent group of one nucleotide to the 3′ hydroxyl or equivalent group of its neighboring nucleotide, optionally via a phosphodiester or other suitable linkage.

A template nucleic acid in exemplary embodiments is a nucleic acid serving as a template for synthesizing a complementary chain in a nucleic acid amplification technique. A complementary chain having a nucleotide sequence complementary to the template has a meaning as a chain corresponding to the template, but the relationship between the two is merely relative. That is, according to the methods described herein a chain synthesized as the complementary chain can function again as a template. That is, the complementary chain can become a template. In certain embodiments, the template is derived from a biological sample, e.g., plant, animal, virus, micro-organism, bacteria, fungus, etc. In certain embodiments, the animal is a mammal, e.g., a human patient.

A template nucleic acid typically comprises one or more target nucleic acid. A target nucleic acid in exemplary embodiments may comprise any single or double-stranded nucleic acid sequence that can be amplified or synthesized according to the disclosure, including any nucleic acid sequence suspected or expected to be present in a sample. In some embodiments, the target sequence is present in double-stranded form and includes at least a portion of the particular nucleotide sequence to be amplified or synthesized, or its complement, prior to the addition of target-specific primers or appended adapters. Target sequences can include the nucleic acids to which primers useful in the amplification or synthesis reaction can hybridize prior to extension by a polymerase. In some embodiments, the term refers to a nucleic acid sequence whose sequence identity, ordering or location of nucleotides is determined by one or more of the methods of the disclosure.

In some embodiments of the invention, a composition (e.g. reaction mixture) having a nucleic acid sample comprising a nucleic acid template is incubated with one or more first amplification primer sets and one or more second primer set(s). In these embodiments, the one or more first primer set(s) and the one or more second primer set(s) compete for binding with the template in and the inclusion of one or more second primer set(s) in the composition reduces non-specific amplification products a NAAT performed according to the invention. Differences between the length, degree of complementarity, and other factors well known in the art may influence the affinity a particular primer has for a particular target nucleic acid or target template, as well as the ability of a particular primer to compete for binding to a template nucleic acid with other primers in the composition or reaction mixture.

Both the first primer pair or set and the second primer pair or set typically have at least a region that is complementary to a nucleic acid template in the sample. NAAT primers used in the compositions, methods, and other inventions described herein typically at least 75% complementary or at least 85% complementary, more typically at least 90% complementary, more typically at least 95% complementary, more typically at least 98% or at least 99% complementary, or identical, to at least a portion of a nucleic acid molecule that includes a target sequence. In such instances, the target primer or target-specific primer and target sequence are described as “corresponding” to each other. In some embodiments, the target-specific primer is capable of hybridizing to at least a portion of its corresponding target sequence (or to a complement of the target sequence); such hybridization can optionally be performed under standard hybridization conditions or under stringent hybridization conditions. In some embodiments, the target-specific primer is not capable of hybridizing to the target sequence, or to its complement, but is capable of hybridizing to a portion of a nucleic acid strand including the target sequence, or to its complement.

In some embodiments, the target-specific primer includes at least one sequence that is at least 75% complementary, typically at least 85% complementary, more typically at least 90% complementary, more typically at least 95% complementary, more typically at least 98% complementary, or more typically at least 99% complementary, to at least a portion of the target sequence itself; in other embodiments, the target-specific primer includes at least one sequence that is at least 75% complementary, typically at least 85% complementary, more typically at least 90% complementary, more typically at least 95% complementary, more typically at least 98% complementary, or more typically at least 99% complementary, to at least a portion of the nucleic acid molecule other than the target sequence. In some embodiments, the target-specific primer is substantially non-complementary to other target sequences present in the sample; optionally, the target-specific primer is substantially non-complementary to other nucleic acid molecules present in the sample. In some embodiments, nucleic acid molecules present in the sample that do not include or correspond to a target sequence (or to a complement of the target sequence) are referred to as “non-specific” sequences or “non-specific nucleic acids”. In some embodiments, the target-specific primer is designed to include a nucleotide sequence that is substantially complementary to at least a portion of its corresponding target sequence. In some embodiments, a target-specific primer is at least 95% complementary, or at least 99% complementary, or identical, across its entire length to at least a portion of a nucleic acid molecule that includes its corresponding target sequence. In some embodiments, a target-specific primer can be at least 90%, at least 95% complementary, at least 98% complementary or at least 99% complementary, or identical, across its entire length to at least a portion of its corresponding target sequence. In some embodiments, a forward target-specific primer and a reverse target-specific primer define a target-specific primer pair that can be used to amplify the target sequence via template-dependent primer extension.

In other embodiments, the primer comprises one or more mismatched nucleotides (i.e., bases that are not complementary to the binding site). In still other embodiments, the primer can comprise a segment that does not anneal to the polynucleic acid or that is complementary to the inverse strand of the polynucleic acid to which the primer anneals. In certain embodiments, a primer is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70 or more nucleotides in length. In a preferred embodiment, the primer comprises from 2 to 100 nucleotides. In some embodiments, primer lengths are in the range of about 10 to about 60 nucleotides, about 12 to about 50 nucleotides, about 15 to about 50 nucleotides, about 18 to 50 nucleotides in length, about 6 to 50 nucleotides in length, about 10 to about 40 nucleotides in length, about 15 to about 40 nucleotides in length, about 18 to 40 nucleotides in length, or a different length. Typically, a primer is capable of hybridizing to a corresponding target sequence and undergoing primer extension when exposed to amplification conditions in the presence of dNTPS and a polymerase. In some instances, the particular nucleotide sequence or a portion of the primer is known at the outset of the amplification reaction or can be determined by one or more of the methods disclosed herein. In some embodiments, the primer includes one or more cleavable groups at one or more locations within the primer.

In some embodiments, the amount of the one or more first primer pair or set is greater than the amount of the second primer pair or set. In representative embodiments, the one or more second primer set(s) is between 5% and 100% of the total of first and second primer sets in the composition or reaction, between 5% and 80% of the total of first and second primer sets in the composition or reaction, between 5% and 60% of the total of first and second primer sets in the composition or reaction, between 5% and 30% of the total of first and second primer sets in the composition or reaction, between 10% and 80% of the total of first and second primer sets in the composition or reaction, between 10% and 50% of the total of first and second primer sets in the composition or reaction, between 10% and 40% of the total of first and second primer sets in the composition or reaction, between 10% and 30% of the total of first and second primer sets in the composition or reaction, between 20% and 70% of the total of first and second primer sets in the composition or reaction, between 30% and 60% of the total of first and second primer sets in the composition or reaction, or between 40% and 50% of the total of first and second primer sets in the composition or reaction (by weight, or molarity).

In alternative embodiments, the one or more second primer set(s) is about 3% of the total of first and second primer sets in the composition or reaction (total primers), about 4% of total primers, about 5% of total primers, about 6% of total primers, about 7% of total primers, about 8% of total primers, about 9% of total primers, about 10% of total primers, about 11% of total primers, about 12% of total primers, about 13% of total primers, about 14% of total primers, about 15% of total primers, about 17% of total primers, about 20% of total primers, about 25% of total primers, about 30% of total primers, about 35% of total primers, about 40% of total primers, about 45% of total primers, about 50% of total primers, about 55% of total primers, about 60% of total primers, about 65% of total primers, about 70% of total primers, about 75% of total primers, about 80% of total primers, about 90% of total primers, or about 95% of total primers.

The particular number and types of primers that are utilized in a certain embodiment will depend on the particular NAAT that is utilized in particular embodiments of suppressing or reducing non-specific amplification products. Particular embodiments comprise reaction mixtures or methods having one (1), two (2), three (3), four (4), five (5), six (6), seven (7), eight (8), nine (9), ten (10),11, 12, 13, 14, 25, 16, 17, 18, 19, 20, or more first amplification primer pairs or sets. Particular embodiments comprise reaction mixtures or methods having one (1), two (2), three (3), four (4), five (5), six (6), seven (7), eight (8), nine (9), ten (10), 11, 12, 13, 14, 25, 16, 17, 18, 19, 20, or more second amplification primer pairs or sets.

One manifestation of LAMP used here requires a total of six primers: two loop-generating primers, two displacement primers and two “Loop primers” (L_{B} and L_{F}). Thus, embodiments of suppressing or reducing non-specific amplification products that utilize or employ LAMP as the NAAT that is optimized will typically use more than one primer pair set and often several primer pair sets. Primer design for LAMP assays thus requires the selection of eight separate regions of a target nucleic acid sequence (the FIP and BIP primers encompass two primer binding sites each), with the BIP/FIP and Loop primers having significant restrictions on their positioning respective to each other. “Loop primers” must be positioned strictly between the B2 and B1 sites and the F2 and F1 sites, respectively, and must be orientated in one particular direction. Further, significant care must be taken in primer design to avoid primer-dimers between the six primers needed (especially difficult as the FIP and BIP primers are generally greater than 40 nucleotides long).

Primers and oligonucleotides used in embodiments herein comprise nucleotides. A nucleotide comprises any compound, including without limitation any naturally occurring nucleotide or analog thereof, which can bind selectively to, or can be polymerized by, a polymerase. Typically, but not necessarily, selective binding of the nucleotide to the polymerase is followed by polymerization of the nucleotide into a nucleic acid strand by the polymerase; occasionally however the nucleotide may dissociate from the polymerase without becoming incorporated into the nucleic acid strand, an event referred to herein as a “non-productive” event. Such nucleotides include not only naturally occurring nucleotides but also any analogs, regardless of their structure, that can bind selectively to, or can be polymerized by, a polymerase. While naturally occurring nucleotides typically comprise base, sugar and phosphate moieties, the nucleotides of the present disclosure can include compounds lacking any one, some or all of such moieties.

In other embodiments, the nucleotide can optionally include a chain of phosphorus atoms comprising three, four, five, six, seven, eight, nine, ten or more phosphorus atoms. In some embodiments, the phosphorus chain can be attached to any carbon of a sugar ring, such as the 5′ carbon. The phosphorus chain can be linked to the sugar with an intervening O or S. In one embodiment, one or more phosphorus atoms in the chain can be part of a phosphate group having P and O. In another embodiment, the phosphorus atoms in the chain can be linked together with intervening O, NH, S, methylene, substituted methylene, ethylene, substituted ethylene, CNH₂, C(O), C(CH₂), CH₂CH₂, or C(OH)CH₂R (where R can be a 4-pyridine or 1-imidazole). In one embodiment, the phosphorus atoms in the chain can have side groups having O, BH3, or S. In the phosphorus chain, a phosphorus atom with a side group other than O can be a substituted phosphate group. In the phosphorus chain, phosphorus atoms with an intervening atom other than O can be a substituted phosphate group. Some examples of nucleotide analogs are described in Xu, U.S. Pat. No. 7,405,281. In some embodiments, the nucleotide comprises a label and referred to herein as a “labeled nucleotide”; the label of the labeled nucleotide is referred to herein as a “nucleotide label”. In some embodiments, the label can be in the form of a fluorescent moiety (e.g. dye), luminescent moiety, or the like attached to the terminal phosphate group, i.e., the phosphate group most distal from the sugar. Some examples of nucleotides that can be used in the disclosed methods and compositions include, but are not limited to, ribonucleotides, deoxyribonucleotides, modified ribonucleotides, modified deoxyribonucleotides, ribonucleotide polyphosphates, deoxyribonucleotide polyphosphates, modified ribonucleotide polyphosphates, modified deoxyribonucleotide polyphosphates, peptide nucleotides, modified peptide nucleotides, metallonucleosides, phosphonate nucleosides, and modified phosphate-sugar backbone nucleotides, analogs, derivatives, or variants of the foregoing compounds, and the like. In some embodiments, the nucleotide can comprise non-oxygen moieties such as, for example, thio- or borano-moieties, in place of the oxygen moiety bridging the alpha phosphate and the sugar of the nucleotide, or the alpha and beta phosphates of the nucleotide, or the beta and gamma phosphates of the nucleotide, or between any other two phosphates of the nucleotide, or any combination thereof. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group can include sulfur substitutions for the various oxygens, e.g. α-thio-nucleotide 5′-triphosphates. For a review of nucleic acid chemistry, see: Shabarova, Z. and Bogdanov, A. Advanced Organic Chemistry of Nucleic Acids, VCH, New York, 1994.

A number of nucleic acid polymerases can be used in the NAATs utilized in certain embodiments provided herein, including any enzyme that can catalyze the polymerization of nucleotides (including analogs thereof) into a nucleic acid strand. Such nucleotide polymerization can occur in a template-dependent fashion. Such polymerases can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze such polymerization. Optionally, the polymerase can be a mutant polymerase comprising one or more mutations involving the replacement of one or more amino acids with other amino acids, the insertion or deletion of one or more amino acids from the polymerase, or the linkage of parts of two or more polymerases. Typically, the polymerase comprises one or more active sites at which nucleotide binding and/or catalysis of nucleotide polymerization can occur. Some exemplary polymerases include without limitation DNA polymerases and RNA polymerases. The term “polymerase” and its variants, as used herein, also includes fusion proteins comprising at least two portions linked to each other, where the first portion comprises a peptide that can catalyze the polymerization of nucleotides into a nucleic acid strand and is linked to a second portion that comprises a second polypeptide. In some embodiments, the second polypeptide can include a reporter enzyme or a processivity-enhancing domain. Optionally, the polymerase can possess 5′ exonuclease activity or terminal transferase activity. In some embodiments, the polymerase can be optionally reactivated, for example through the use of heat, chemicals or re-addition of new amounts of polymerase into a reaction mixture. In some embodiments, the polymerase can include a hot-start polymerase or an aptamer-based polymerase that optionally can be reactivated.

In certain embodiments of the invention, a multiplexed nucleic acid amplification and real-time detection method is provided. An exemplary embodiment of such a method comprises the steps of i) providing a composition comprising a target nucleic acid sample comprising a template having a region of interest, one or more first amplification primer sets, one or more second primer sets, a polymerase, and deoxynucleotide triphosphates; ii) performing a first reaction to amplify the region of interest, thereby forming a primary amplicon; iii) dividing (ii) into at least two secondary reactions, and including in at least one of the reactions one or more site-specific secondary primer that is complementary to a site-specific primer binding site that may be present within the primary amplicon and defines a site of interest within the region of interest; iv) performing a second reaction (second-stage reaction) thereby accelerating the amplification of the region of interest only if the site-specific primer binding site is complementary to the site-specific primer; and v) detecting and comparing the amplification rates of the at least two secondary reactions, wherein the one or more first primer sets and the one or more second primer sets compete for binding with the template, and the inclusion of one or more second primer sets in the composition reduces non-specific amplification products when the template is amplified, and wherein an enhanced relative rate of amplification in the reaction with the secondary primer indicates the presence of the site of interest that is complementary to the secondary primer. Still further embodiments include apparatus and systems adapted to perform a multiplexed nucleic acid amplification and/or real-time detection methods such as the above and other methods described herein used in conjunction with variations of the above method.

An apparatus described herein or otherwise known in the art can be used as components for the assembly of systems, including those designed to perform methods of the invention. One non-limiting embodiment of a system for performing nucleic acid amplification comprises: i) a central chamber for performing an amplification reaction of an amplification composition or reaction mixture, said amplification reaction mixture comprising a) a nucleic acid sample comprising a template; b) one or more first amplification primer set(s); c) one or more second primer set(s); d) a polymerase; and e) deoxynucleotide triphosphates, wherein during an amplification reaction performed in the system, the one or more first primer set(s) and the one or more second primer set(s) compete for binding with the template and the inclusion of one or more second primer set(s) in the composition reduces non-specific amplification products wherein, optionally, the central chamber is in communication with one or more ii) additional chambers, in which, one or more additional amplification reactions takes place; iii) an instrument for detecting and comparing in real-time the amplification rates of the at least two secondary reactions; and optionally iv) a reaction mixture comprising reagents for performing a nucleic acid amplification and real-time detection method in the system.

Other aspects of the invention may be described in the follow exemplary embodiments:
1. A composition comprising: a) a nucleic acid sample comprising a template; b) one or more first amplification primer sets; c) one or more second primer sets; d) a polymerase; and e) deoxynucleotide triphosphates, wherein the composition is capable of amplifying the template when placed under amplification conditions, wherein the one or more first primer set(s) and the one or more second primer set(s) compete for binding with the template, and the inclusion of one or more second primer sets in the composition reduces non-specific amplification products when the template is amplified.
2. A composition of embodiment 1, wherein the composition is reaction mixture.
3. A composition of embodiment 1 or 2, wherein the template nucleic acid sample comprises a target nucleic acid.
4. A composition of embodiment 1 or 2, wherein the template nucleic acid sample is genomic DNA.
5. A composition of embodiment 1 or 2, wherein the one or more first primer set(s) is between about 10 and about 60 nucleotides in length.
6. A composition of embodiment 1 or 2, wherein the one or more first primer set(s) is between about 18 and about 50 nucleotides in length.
7. A composition of embodiment 1 or 2, wherein the one or more second primer set(s) is between about 6 and about 50 nucleotides in length.
8. A composition of embodiment 1 or 2, wherein the one or more second primer set(s) is between about 6 and about 12 nucleotides in length.
9. A composition of embodiment 1 or 2, wherein the one or more first primer set(s) is greater in length than the one or more second primer set(s).
10. A composition of embodiment 1 or 2, wherein the at least one second primer set has one or more mismatched nucleotide with the template.
11. A composition of embodiment 1 or 2, wherein the one or more second primer set(s) has two mismatched nucleotides with the template.
12. A composition of embodiment 1 or 2, wherein the one or more first primer set(s) have a higher binding affinity for the template in the composition than the one or more second primer set(s).
13. A composition of embodiment 1 or 2, wherein the one or more second primer set(s) comprises modified or non-natural nucleotide analogs.
14. A composition of embodiment 1 or 2, wherein the one or more second primer set(s) comprises one or more modification relative to unmodified nucleic acid which increase nuclease resistance.
15. A composition according to embodiment 14, wherein said one or more second primer set are resistant to the 3′ proof reading activity of a DNA polymerase.
16. A composition of embodiment 1 or 2, wherein the one or more second primer set(s) has a modified 3′ terminal nucleotide.
17. A composition of embodiment 16, wherein upon amplification said modified 3′ terminal nucleotide reduces the amount of amplification of products comprising the one or more second primer sets relative to the amount of amplification of products comprising the one or more first primer sets.
18. A composition of embodiment 1 or 2, wherein the one or more second primer set(s) is between 5% and 100% of the total of first and second primer sets in the composition, between 5% and 80% of the total of first and second primer sets in the composition, between 5% and 60% of the total of first and second primer sets in the composition, between 5% and 30% of the total of first and second primer sets in the composition, between 10% and 80% of the total of first and second primer sets in the composition, between 10% and 50% of the total of first and second primer sets in the composition, between 10% and 40% of the total of first and second primer sets in the composition, between 10% and 30% of the total of first and second primer sets in the composition, between 20% and 70% of the total of first and second primer sets in the composition, between 30% and 60% of the total of first and second primer sets in the composition, or between 40% and 50% of the total of first and second primer sets in the composition.
19. A composition of embodiment 1 or 2, wherein the one or more second primer set(s) is between 15% and 100% of the total percentage by molarity of first and second primer sets in the composition.
20. A composition of embodiment 1 or 2, wherein the polymerase is selected from a strand-displacing polymerase, BstL, BstX, phi29, Bsu, Taq, Klentaq, KOD, KOD exo(-), and Phusion.
21. A composition of embodiment 2, wherein the reaction mixture is an amplification reaction mixture selected from a loop-mediated (LAMP) reaction mixture, stand displacement reaction mixture (SDS), Polymerase Chain Reaction (PCR), a ligase chain reaction (LCR), Isothermal Chimeric Amplification of Nucleic Acids (ICAN), SMart Amplification Process (SMAP), Chimeric Displacement Reaction (RDC), (exponential)-rolling circle amplification (exponential-RCA), Nucleic Acid Sequence Based Amplification (NASBA), Transcription Mediated Amplification (TMA), Helicase Dependent Amplification (HDA) and Recombinase polymerase amplification (RPA), and Cross Primed Amplification (CPA).
22. A kit for detecting or quantifying a target nucleic acid in a nucleic acid sample, the kit comprising a composition according to any one of embodiments 1-20 and instructions for use.
23. A multiplexed nucleic acid amplification and real-time detection method comprising:

a. providing a composition comprising a target nucleic acid sample comprising a template having a region of interest, one or more first amplification primer sets, one or more second primer sets, a polymerase, and deoxynucleotide triphosphates; b. performing a first reaction to amplify the region of interest, thereby forming a primary amplicon; c. dividing (b) into at least two secondary reactions, and including in at least one of the reactions one or more site-specific secondary primer that is complementary to a site-specific primer binding site that may be present within the primary amplicon and defines a site of interest within the region of interest; d. performing a second reaction (second-stage reaction) thereby accelerating the amplification of the region of interest only if the site-specific primer binding site is complementary to the site-specific primer; and e. detecting and comparing the amplification rates of the at least two secondary reactions, wherein an enhanced relative rate of amplification in the reaction with the secondary primer indicates the presence of the site of interest that is complementary to the secondary primer, and

wherein the one or more first primer set(s) and the one or more second primer set(s) compete for binding with the template in step b) and/or step d), and

the inclusion of one or more second primer set(s) in the composition reduces non-specific amplification products.
24. A method according to embodiment 23, wherein the amplification conditions are isothermal amplification conditions.
25. A method according to embodiment 23, wherein the amplification conditions comprise thermocycling.
26. A method according to embodiment 23, wherein the method further comprises quantifying the amount of amplified template.
27. A method of detecting or quantifying a target nucleic acid in a nucleic acid sample and reducing the amplification of non-template molecules from the sample, the method comprising: i)

incubating a composition comprising a nucleic acid sample comprising a template; one or more first amplification primer set(s); one or more second primer set(s); a polymerase; and deoxynucleotide triphosphates, ii) amplifying the template by an isothermal NAAT, wherein the one or more first primer set(s) and the one or more second primer set(s) compete for binding with the template in step i) and/or step ii), and the inclusion of one or more second primer set(s) in the composition reduces non-specific amplification products in step ii); and iii) quantifying the amount of amplified template.
28. A system for performing an amplification reaction, comprising:

i) a central chamber for performing an amplification reaction of an amplification composition or reaction mixture, said amplification reaction mixture comprising a) a nucleic acid sample comprising a template; b) one or more first amplification primer set(s); c) one or more second primer set(s); d) a polymerase; and e) deoxynucleotide triphosphates, wherein during an amplification reaction performed in the system, the one or more first primer set(s) and the one or more second primer set(s) compete for binding with the template and the inclusion of one or more second primer set(s) in the composition reduces non-specific amplification products wherein, optionally, the central chamber is in communication with one or more ii) additional chambers, in which, one or more additional amplification reactions takes place; iii) an instrument for detecting and comparing in real-time the amplification rates of the at least two secondary reactions; and optionally iv) a reaction mixture comprising reagents for performing a nucleic acid amplification and real-time detection method in the system.

The following Examples are included for illustration and not limitation.

### EXAMPLE 1

LAMP primer reactions consisting of F3, B3, FIP, BIP and a STEM primer were used with the TangenDx instrument using 600 copies of target genomic DNA (Templated True Positive) or with no copies (No-Template False Positive) of the genomic target. Blocking oligos with sequences identical to F3, B3, BIP, FIP and the STEM primer were synthesized with a 3-carbon spacer blocking the 3′ end (Integrated DNA technologies) and the amount of total blocking oligo was varied from 15-25% compared to unblocked primers. The Cq data recorded is the output from the TangenDx instrument and is defined as the number of cycles before a positive reaction is quantitated. Each cycle is 15 seconds (40 cycles is 10 minutes)

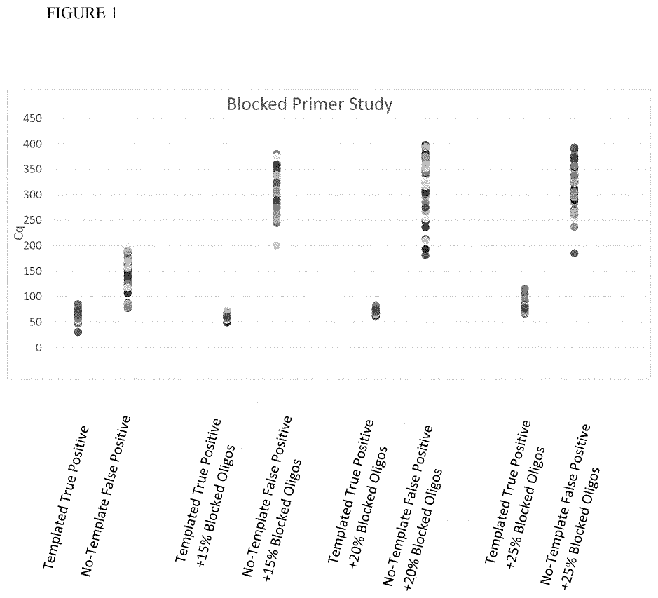

In FIG. 1. there was an overlap in timing of the appearance of slow true positives with fast false positives. The reactions containing an additional >15% blocked oligo (blocked LAMP oligos contained a 3′ 3-carbon spacer) shifted the timing of the appearance of NTFP such that the gap between the fastest NTFP and the TTPs was >130 Cq (˜30 Minutes) without impacting the timing of the appearance of the TTPs compared to the unblocked condition.

### EXAMPLE 1I

LAMP reactions conditions: LAMP primers for C_alb were designed targeting the ITS2 region of the 18s rRNA gene of Candida albicans (ATCC 90028), the KPC2 gene of Klebsiella pnemoniae (ATCC BAA-1705), and the vanA gene of Enterococcus faecium (ATCC® 700221DQ). The basic LAMP reactions contained the primers C_alb1, KPC2f, or VanA5 (Refer to table 2 for the sequences and LAMP reaction concentrations). The full-length 3′ phosphate blocked primers, C_alb1_P, KPC2f_P, VanA5_P (Refer to table 2 for the sequences) were added in addition to the existing concentration of unblocked primers at varying concentrations to the basic LAMP reaction primer set where indicated. The truncated primers, C_alb1_8merP, KPC2fs1_8merP, VanA5_8merP consisting of 3′ phosphate 8mers of the first 8 nucleotides of the 3′ end of the primer sequence were added in addition to the existing concentration of unblocked primers at varying concentrations to the basic LAMP reaction primer set where indicated (Refer to table 2 for the sequences). The LAMP pre-mix was prepared on ice with 100 mM dNTP mix, 267× EVA Green™qPCR dye, 1M MgSO4, 1% TritonX (detergent, and TIB3 buffer (Tangen isotheuaal Buffer 3) containing the following components; KCl, Tris-I-HCl pH18.8, (NH4)2SO4-, Brij-35, and glycerol. The pre-mix was stored at −20 degree C. and it was thawed at room temperature prior to use. The LAMP reaction was assembled by combing the pre-mix, LAMP primers (either unblocked or a blend of both unblocked and blocked primers, and a positive control, MS14 containing 2000 genomes of Mycobacterium smegmatis DNA), BST2.0 (New England Biolabs) (Refer to Table 1 for the final concentration of each component), and C. albicans DNA (2000 genomes per 25 uL reaction) or buffer for the positive control or negative control, respectively. The mixture was pipetted using a multi-channel pipet⁻tor into a 96-well plate (Bio-Rad Hard Shell® PCR Plates white/clear 96 wells, HSP9601), and sealed with Thermaseal RT™ sealing films (TS-RT2-100 Non-sterile), with the following format: 4 wells contain positive controls for the LAMP reaction, MS14. 4 positive controls for C_alb1 containing 2000 C. albicans genomes. The remaining 88 wells for C_alb1 negative control containing no template DNA. The plate was put into the Bio-Rad CFX Connect Real-Time system (SN:788BR02205). The following steps were run. Start at 4.0° C. for 60 seconds, then ramp to 66° C. The entire plate was read every 15 seconds for a total of 300 cycles. At the end of the run, a melt of curve was generated with a profile from 66.0° C. to 95.0° C. with a ramp of 0.5° C. per 5 seconds.

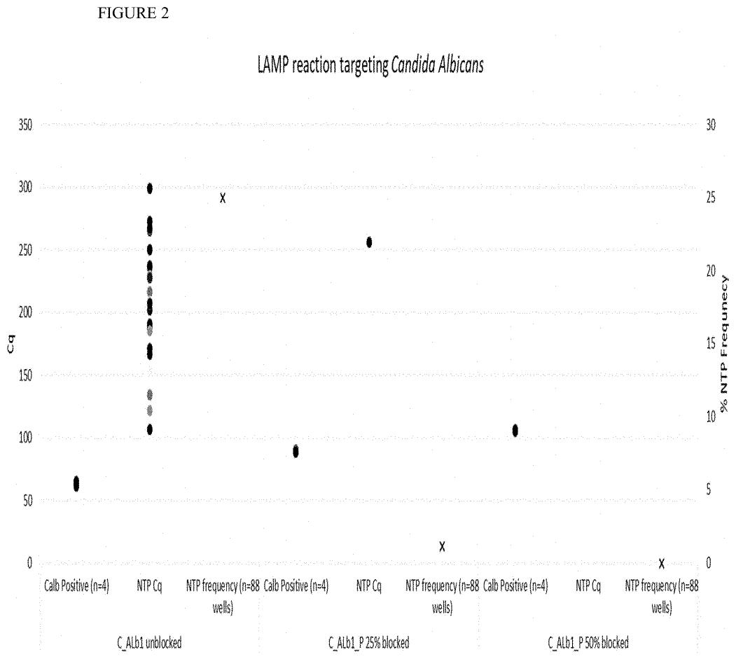

For all three of the LAMP reactions studied (C_alb1, vanA5 and KPC2f), the addition of unextendable 3′ blocked primers containing identical sequences to the active LAMP primers had the effect of disproportionately inhibiting the timing of formation of non-template products (NTPs) and their frequency compared to Template Positive reactions (TP), in a concentration dependent manner. The full-length blocking primers also influencing the speed of the correctly templated true positive reactions (FIGS. 2-5). For example, the C. alb unblocked reaction had an NTP frequency of 25% (n=88 wells) with the fastest NTP at a Cq (quantification cycle, 1 cycle=15 seconds) of 106. The slowest C. alb TP was observed at a Cq of 65. The window between the fastest NTP and slowest TP of the C. alb unblocked run was 41. With the addition of 25% full length blocking primers, C_Alb1_P, the NTP frequency decreased from 25% to 1.1%. The Cq window increased to 166, with the fastest NTP at 256, and the slowest TP at 90. Further increasing the concentration of the full-length blocked primer to 50% decreased the NTP frequency to 0% in 300 cycles. At a concentration of 50% full length blocking primers, C_Alb1_P, the C. alb true positives slowed to an average of 105.6±0.7% with the slowest at 106 (FIG. 2). Overall, the full-length blocking group greatly decreased the NTP Frequency and NTP speed of formation, at the cost of slowing down the TP reactions.

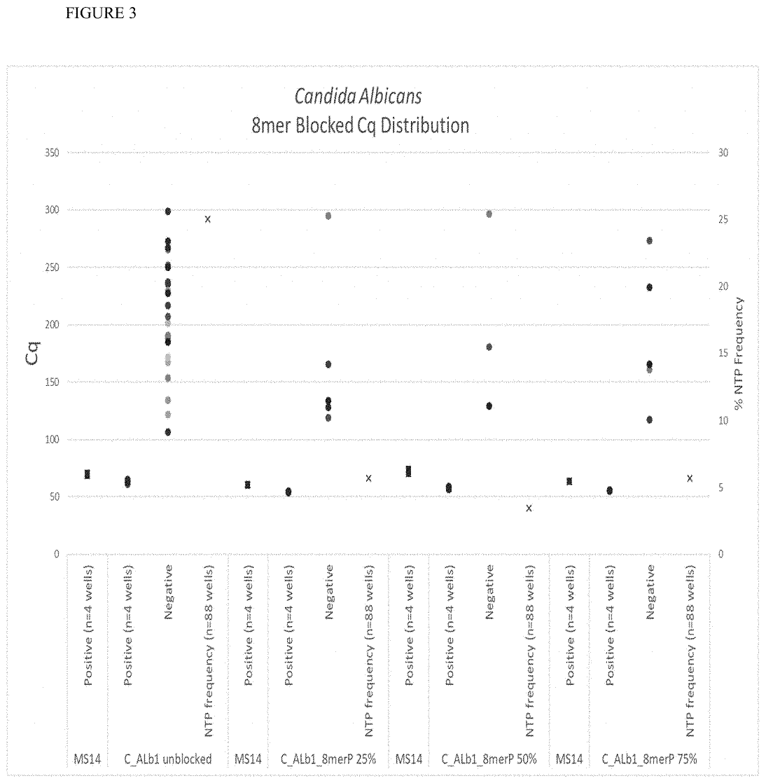

Truncated Blocked primers containing identical sequences to the 8 terminal 3′ nucleotides of the full length active LAMP primers and containing 3′ ends that were unable to be extended by a DNA polymerase (8merP), were also able to inhibit NTP frequency and speed, however, these 8mer 3′blocked oligos did not inhibit the TP reactions (FIG. 3).

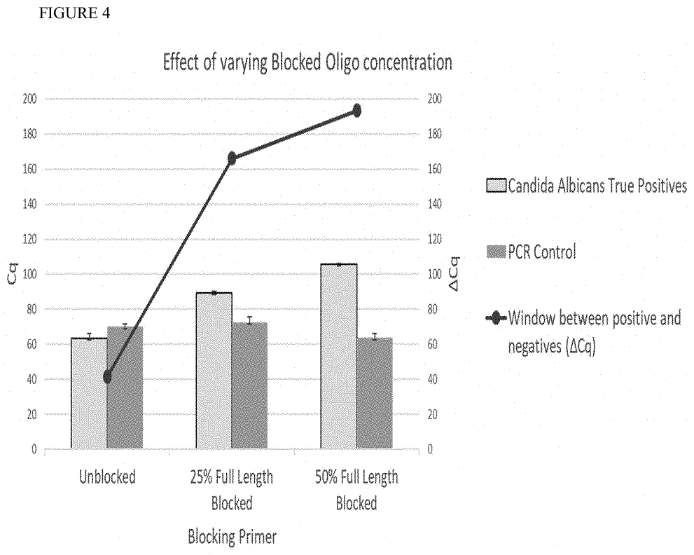

In general, the full-length blocked primers showed greater efficacy for inhibiting the timing of formation of NTPs and their frequency compared to the 8merP counterparts. The magnitude of the inhibition of NTPs is expressed as the difference between the slowest TP LAMP reaction (2000 copy input of target genome) and the fastest NTP product Cq and is shown for the three LAMP reaction primer sets C_alb1, vanA5 and KPC2f (Table 3 and FIGS. 2 and 4 and figure legends).

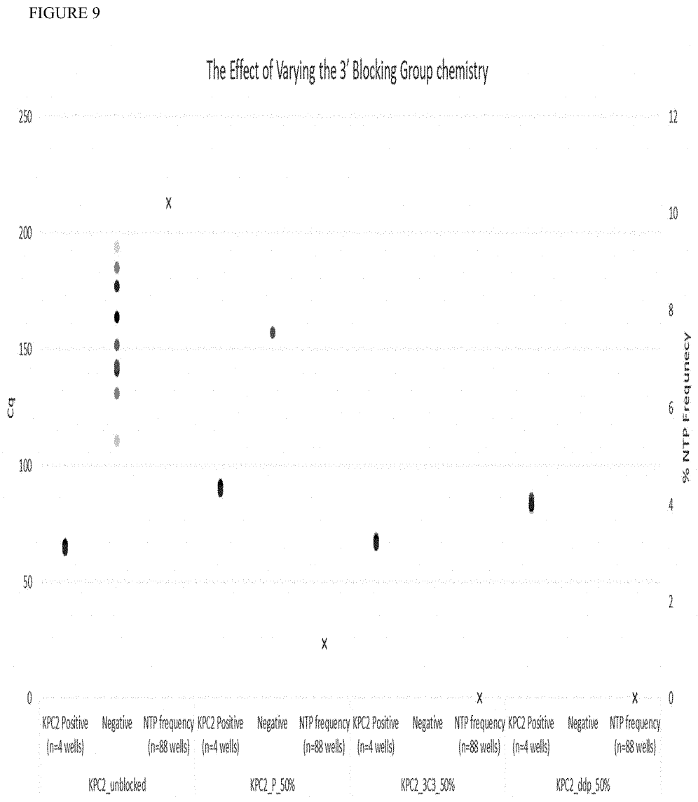

Increasing the concentration of full length 3′ blocked primers over the range of 0 to 50% in each case further widened the gap between TP and NTP, while the 8-merP blocking primers showed reduced effects that exhibited varying degrees of efficacy between the three primer sets. In the case of the C_alb1 LAMP primer reactions, increasing the 8merP blocking % through the range 0, 25%, 50%, 75% showed a plateau of inhibition of timing and formation of NTPs indicating that a small fraction of NTPs were resistant to the inhibiting action of the 8merP oligos. Since the 8merP oligos compete with the 3′ ends of active primers, and do not block the 5′ ends of the active primers, it is possible that active priming events occurring at regions that hybridize to the 5′ end of unblocked primer sequences generate extended primers that have new sequences at the 3′ end that can participate in the formation of NTPs and are no longer in competition with the 8merP blocking sequences. Since the specific NTP species that form are entirely sequence dependent, different LAMP primer set show differing responses to the truncated 8merP blocking oligos. The full length 3′ blocked oligos were able to inhibit the formation of NTPs across the entire length of their sequence space. Other blocking groups were shown to have similar effects to a 3′ phosphate, including a 3′ terminal dideoxy base and a 3 carbon spacer at the 3′ end (see FIG. 9 below).

TABLE 1
Final concentration of LAMP reaction components in a 25 uL reaction

| | Product Information | Un-blocked | | 25% blocked | | 50% blocked | | 75% blocked | |
|---|---|---|---|---|---|---|---|---|---|
| EvaGreen | Biotium Cat #31019 | 1x | | 1x | | 1x | | 1x | |
| Warm Start BST 2.0 | NEB Cat# M0538L | 24 | units | 24 | units | 24 | units | 24 | units |
| Triton X-100 | Sigma Cat# X100 | 0.01% | | 0.01% | | 0.01% | | 0.01% | |
| dNTPs | MyChem cat # MCL- | 2.5 | mM | 2.5 | mM | 2.5 | mM | 2.5 | mM |
| | 1000-100 | | | | | | | | |
| MgSO4 | Alfa Aesar Cat #J601030 | 3.3 | mM | 3.3 | mM | 3.3 | mM | 3.3 | mM |
| Tris-HCl, pH 8.8 | Corning Cat# 46-031-CM | 18.8 | mM | 18.8 | mM | 18.8 | mM | 18.8 | mM |
| (NH4)2SO4— | Sigma Cat# A4418 | 7.8 | mM | 7.8 | mM | 7.8 | mM | 7.8 | mM |
| KCl | Quality Biological Cat# | 54.7 | mM | 54.7 | mM | 54.7 | mM | 54.7 | mM |
| | 351-044-101 | | | | | | | | |
| Brij-35 | Thermo Fisher Cat# 28316 | 0.1% | | 0.1% | | 0.1% | | 0.1% | |
| Glycerol | MP Bio Cat# 800688 | 7.8% | | 7.8% | | 7.8% | | 7.8% | |
| Unblocked_STEM | IDT | 1.65 | uM | 1.65 | uM | 1.65 | uM | 1.65 | uM |
| Unblocked_F3 | IDT | 0.1 | uM | 0.1 | uM | 0.1 | uM | 0.1 | uM |
| Unblocked_B3 | IDT | 0.1 | uM | 0.1 | uM | 0.1 | uM | 0.1 | uM |
| Unblocked_FIP | IDT | 1.65 | uM | 1.65 | uM | 1.65 | uM | 1.65 | uM |
| Unblocked_BIP | IDT | 1.65 | uM | 1.65 | uM | 1.65 | uM | 1.65 | uM |
| Blocked_STEM | IDT | 0 | uM | 0.41 | uM | 0.83 | uM | 1.24 | uM |
| Blocked_F3 | IDT | 0 | uM | 0.03 | uM | 0.05 | uM | 0.08 | uM |
| Blocked_B3 | IDT | 0 | uM | 0.03 | uM | 0.05 | uM | 0.08 | uM |
| Blocked_FIP | IDT | 0 | uM | 0.41 | uM | 0.83 | uM | 1.24 | uM |
| Blocked_BIP | IDT | 0 | uM | 0.41 | uM | 0.83 | uM | 1.24 | uM |

TABLE 2
Primer sequences for KPC2f, C_alb1, and VanA5.

| Primer Name | Sequence |
|---|---|
| KPC2f_F3 | GGCGGAGTTCAGCTCCAG (SEQ ID NO: 1) |
| KPC2f_B3 | CCGTTACGGCAAAAATGCG (SEQ ID NO: 2) |
| KPC2f_FIP | CTGAAGGAGTTGGGCGGCCCGTCCAGACGGAACGTGGTA (SEQ ID NO: 3) |
| KPC2f_BIP | TGTATTGCACGGCGGCCGGIGGTCACCCATCTCGGAA (SEQ ID NO: 4) |
| KPC2f_Stem1 | AATTGGCGGCGGCGTTAT (SEQ ID NO: 5) |
| KPC2f_F3_P | GGCGGAGTICAGCTCCAG/3Phos/ (SEQ ID NO: 6) |
| KPC2f_B3_P | CCGTTACGGCAAAAATGCG/3Phos/ (SEQ ID NO: 7) |
| KPC2f_FIP_P | CTGAAGGAGTTGGGCGGCCCGTCCAGACGGAACGTGGTA/3Phos/ (SEQ |
| | ID NO: 8) |
| KPC2f_BIP_P | TGTATTGCACGGCGGCCGGIGGTCACCCATCTCGGAA/3Phos/ (SEQ ID |
| | NO: 9) |
| KPC2f_Stem1_P | AATTGGCGGCGGCGTTAT/3Phos/ (SEQ ID NO: 10) |
| KPC2f_F3_8merP | AGCTCCAG/3Phos/ (SEQ ID NO: 11) |
| KPC2f_B3_8merP | AAAATGCG/3Phos/ (SEQ ID NO: 12) |
| KPC2f_FIP_8merP | ACGTGGTA/3Phos/ (SEQ ID NO: 13) |
| KPC2f_BIP_8merP | TCTCGGAA/3Phos/ (SEQ ID NO: 14) |
| KPC2f_Stem1_8merP | GGCGTTAT/3Phos/ (SEQ ID NO: 15) |
| KPC2f_F3_10merP | TCAGCTCCAG/3Phos/ (SEQ ID NO: 16) |
| KPC2f_B3_10merP | CAAAAATGCG/3Phos/ (SEQ ID NO: 17) |
| KPC2f_FIP_10merP | GAACGTGGTA/3Phos/ (SEQ ID NO: 18) |
| KPC2f_BIP_10merP | CATCTCGGAA/3Phos/ (SEQ ID NO: 19) |
| KPC2f_Stem1_10merP | CGGCGTTAT/3Phos/ (SEQ ID NO: 20) |
| KPC2f_F3_12merP | GTTCAGCTCCAG/3Phos/ (SEQ ID NO: 21) |
| KPC2f_B3_12merP | GGCAAAAATGCG/3Phos/ (SEQ ID NO: 22) |
| KPC2f_FlP_12merP | CGGAACGTGGTA/3Phos/ (SEQ ID NO: 23) |
| KPC2f_BIP_12merP | CCCATCTCGGAA/3Phos/ (SEQ ID NO: 24) |
| KPC2f_Stem1_12merP | GGCGGCGTTAT/3Phos/ (SEQ ID NO: 25) |
| KPC2f_F3_3C3 | GGCGGAGTTCAGCTCCAG/3SpC3/ (SEQ ID NO: 26) |
| KPC2f_B3_3C3 | CCGTTACGGCAAAAATGCG/3SpC3/ (SEQ ID NO: 27) |
| KPC2f_FIP_3C3 | CTGAAGGAGTTGGGCGGCCCGTCCAGACGGAACGTGGTA/3SpC3/ (SEQ |
| | ID NO: 28) |
| KPC2f_BIP_3C3 | TGTATTGCACGGCGGCCGGTGGTCACCCATCTCGGAA/3SpC3/ (SEQ ID |
| | NO: 29) |
| KPC2f_Stem1_3C3 | AATTGGCGGCGGCGTTAT/3SpC3/ (SEQ ID NO: 30) |
| KPC2f_F3_ddc | GGCGGAGTTCAGCTCCAG/3ddC/ (SEQ ID NO: 31) |
| KPC2f_B3_ddc | CCGTTACGGCAAAAATGCG/3ddC/ (SEQ ID NO: 32) |
| KPC2f_FIP_ddc | CTGAAGGAGTTGGGCGGCCCGTCCAGACGGAACGTGGTA/3ddC/ (SEQ |
| | ID NO: 33) |
| KPC2f_BIP_ddc | TGTATTGCACGGCGGCCGGTGGTCACCCATCTCGGAA/3ddC/ (SEQ ID |
| | NO: 34) |
| KPC2f_Stem1_ddc | AATTGGCGGCGGCGTTAT/3ddC/ (SEQ ID NO: 35) |
| C_alb1_F3 | AGCGTCGTTTCTCCCTCAA (SEQ ID NO: 36) |
| C_alb1_B3 | TCCTCCGCTTATTGATATGCTT (SEQ ID NO: 37) |
| C_alb1_FIP | CGCCTTACCACTACCGTCTTTCACCGCTGGGTTTGGTGTTG (SEQ ID |
| | NO: 38) |
| C_alb1_BIP | TAACCAAAAACATTGCTTGCGGCGGCGGGTAGTCCTACCTGAT (SEQ ID |
| | NO: 39) |
| C_alb1_STEM | GACCTAAGCCATTGTCAAAGCGATC (SEQ ID NO: 40) |
| C_alb1_F3_P | AGCGTCGTTTCTCCCTCAA/3Phos/ (SEQ ID NO: 41) |
| C_alb1_B3_P | TCCTCCGCTTATTGATATGCTT/3Phos/ (SEQ ID NO: 42) |
| C_alb1_FIP_P | CGCCTTACCACTACCGTCTTTCACCGCTGGGTTTGGTGTTG/3Phos/ (SEQ |
| | ID NO: 43) |
| C_alb1_BIP_P | TAACCAAAAACATTGCTTGCGGCGGCGGGTAGTCCTACCTGAT/3Phos/ |
| | (SEQ ID NO: 44) |
| C_alb1_STEM_P | GACCTAAGCCATTGTCAAAGCGATC/3Phos/ (SEQ ID NO: 45) |
| C_alb1_F3_8merP | TCCCTCAA/3Phos/ (SEQ ID NO: 46) |
| C_alb1_B3_8merP | ATATGCTT/3Phos/ (SEQ ID NO: 47) |
| C_alb1_FIP_8merP | TGGTGTTG/3Phos/ (SEQ ID NO: 48) |
| C_alb1_BIP_8merP | TACCTGAT/3Phos/ (SEQ ID NO: 49) |
| C_alb1_STEM_8merP | AAGCGATC/3Phos/ (SEQ ID NO: 50) |
| vanA5_F3 | CGCAATTGAATCGGCAAGAC (SEQ ID NO: 51) |
| vanA5_B3 | CCTCGCTCCTCTGCTGAA (SEQ ID NO: 52) |
| vanA5_FIP | ACGCGGCACTGTTTCCCAATACAATTGAGCAGGCTGTTTCGG (SEQ ID |
| | NO: 53) |
| vanA5_BIP | TTCATCAGGAAGTCGAGCCGGAGGTCTGCGGGAACGGTTA (SEQ ID |
| | NO: 54) |
| vanA5_Stem2 | GTACTGCAGCCTGATTTGGTCC (SEQ ID NO: 55) |
| vanA5_F3_P | CGCAATTGAATCGGCAAGAC/3Phos/ (SEQ ID NO: 56) |
| vanA5_B3_P | CCTCGCTCCTCTGCTGAA/3Phos/ (SEQ ID NO: 57) |
| vanA5_FIP_P | ACGCGGCACTGTTTCCCAATACAATTGAGCAGGCTGTTTCGG/3Phos/ |
| | (SEQ ID NO: 58) |
| vanA5_BIP_P | TTCATCAGGAAGTCGAGCCGGAGGTCTGCGGGAACGGTTA/3Phos/ (SEQ |
| | ID NO: 59) |
| vanA5_Stem2_P | GTACTGCAGCCTGATTTGGTCC/3Phos/ (SEQ ID NO: 60) |
| vanA5_F3_8merP | GGCAAGAC/3Phos/ (SEQ ID NO: 61) |
| vanA5_B3_8merP | CTGCTGAA/3Phos/ (SEQ ID NO: 62) |
| vanA5_FIP_8merP | TGTTTCGG/3Phos/ (SEQ ID NO: 63) |
| vanA5_BIP_8merP | AACGGTTA/3Phos/ (SEQ ID NO: 64) |
| vanA5_Stem2_8merP | TTTGGTCC/3Phos/ (SEQ ID NO: 65) |

TABLE 3
LAMP reaction conditions for LAMP primer set C_alb1
shown with corresponding window between the slowest C. Albicans
true positive well and the fastest NTP well.

| C_alb1 | Cq window between positives and negatives |
|---|---|
| Unblocked | 41 |
| 25% Full length blocked | 166 |
| 50% Full length blocked | 193 |
| 25% 8merP | 63 |
| 50% 8merP | 69 |
| 75% 8merP | 61 |

In the experiment shown in FIG. 4, blocked oligos (3′ phosphate) were added to a standard LAMP reactions to test their ability to reduce the formation of no template false positives during amplification. The Y axis shows the Cq gap (ΔCq) between the slowest true positive and the fastest no-template false positive. As can be seen in the data in Table 3, the Cq gap increases from about 41to about 166 in a reaction where 25% of the full-length primers are blocked and again that the Cq gap, or differential between positives and negatives, increases to about 193 in the reaction where 50% of the full-length primers are blocked.

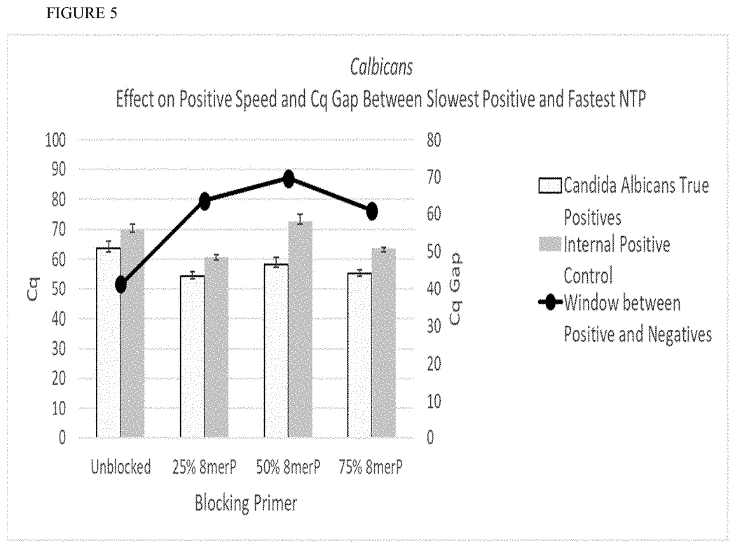

In the experiment shown having data presented in Table 3 and in FIG. 5 the effect of varying blocked oligonucleotide concentration in Candida albicans is shown. C_alb1 is a LAMP primer set that targets the 18s rRNA gene of Candida albicans. The X axis identifies each concentration of C_alb_8merP (blocked), tested as well as the control with no C_alb_8merP (unblocked). The Y axis identifies the Cq (quantification cycle, 1 cycle=15 seconds) or speed of the reaction, the secondary Y axis identifies the Cq gap between the slowest true positive and the fastest no-template positive well. As can be seen in Table 3, the Cq gap increases from about 41 in unblocked, to about 63 in a reaction where 25% of the 8mer primers are blocked and again that the Cq gap increases to about 69 in the reaction where 50% of the 8mer primers are blocked. However, in this experiment the Cq gap was about 61 in the reaction where 75% of the 8mer primers are blocked, which is lower than the 50% reaction.

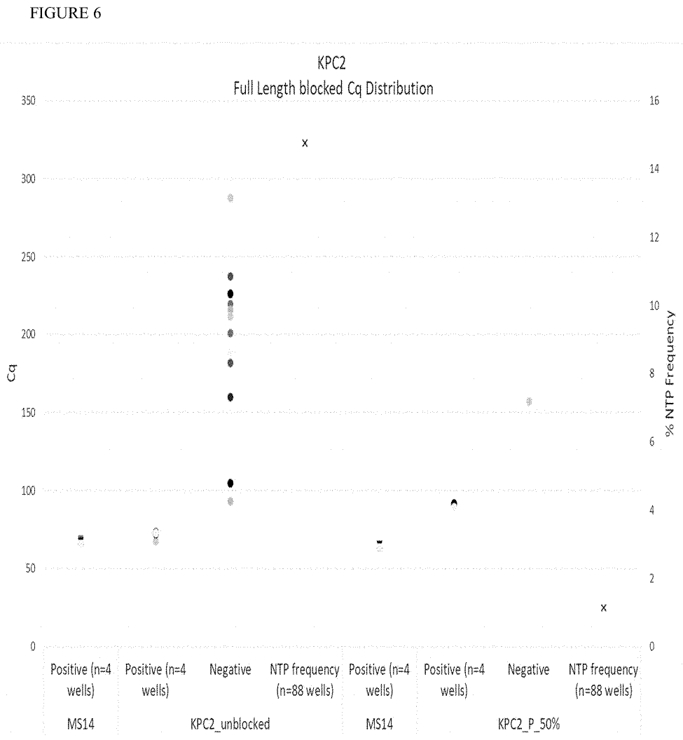

In the experiment shown in FIG. 6. KPC2f is a LAMP primer set that targets the KPC2 gene of Klebsiella pneumoniae. The X axis identifies the distribution of true positives (2000 genomes Klebsiella pneumoniae (KPC2+)) and negatives wells on a 96 well qPCR Plate for each condition of tested while the Y axis identifies the Cq (quantification cycle, 1 cycle=15 seconds) or speed of the reaction. The secondary Y axis identifies the % NTP frequency which is the frequency of wells showing NTPs within 300 scans. At a concentration where the blocked primer (full length primer with a 3′ Phosphate blocking group, KPC2f P) was added at 50% in addition to the 100% standard unblocked oligo concentration, the addition of the blocking primers resulted in a reduction of NTPs from 15% to around 1%. The positive control templated positive reactions were slowed from a Cq of 72 to 90, while the Cq differential between TP and NTP increased from 21 to 67.

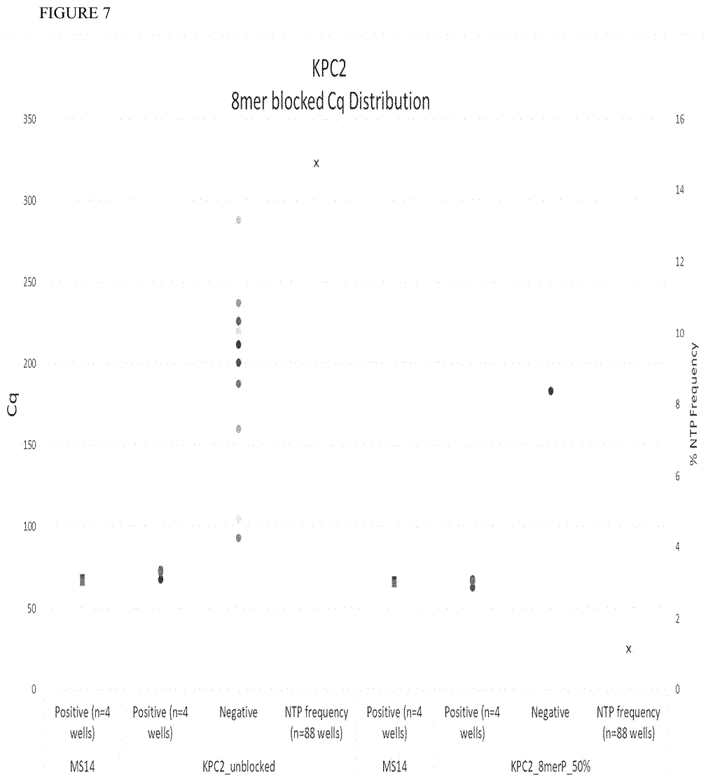

In the experiment shown in FIG. 7. KPC2f is a LAMP primer set that targets the KPC2 gene of Klebsiella pneumoniae. The X axis identifies the distribution of positives and negatives wells on a 96 well qPCR Plate for each condition of tested while the Y axis identifies the Cq (quantification cycle, 1 cycle=15 seconds) or speed of the reaction. The secondary Y axis identifies the % NTP frequency which is the frequency of wells showing NTPs within 300 scans. At a concentration where the blocking primers (8-mer oligos with a 3′ Phosphate blocking group, KPC2fs1_8mer_P) was added at 50% in addition to the 100% standard unblocked oligo concentration , the addition of the 8-mer blocking primers resulted in a reduction of NTPs from 15% to around 1%. The positive control template positive reactions remained unchanged from a Cq of 72, while the Cq differential between TP and NTP increased from 18 to 108

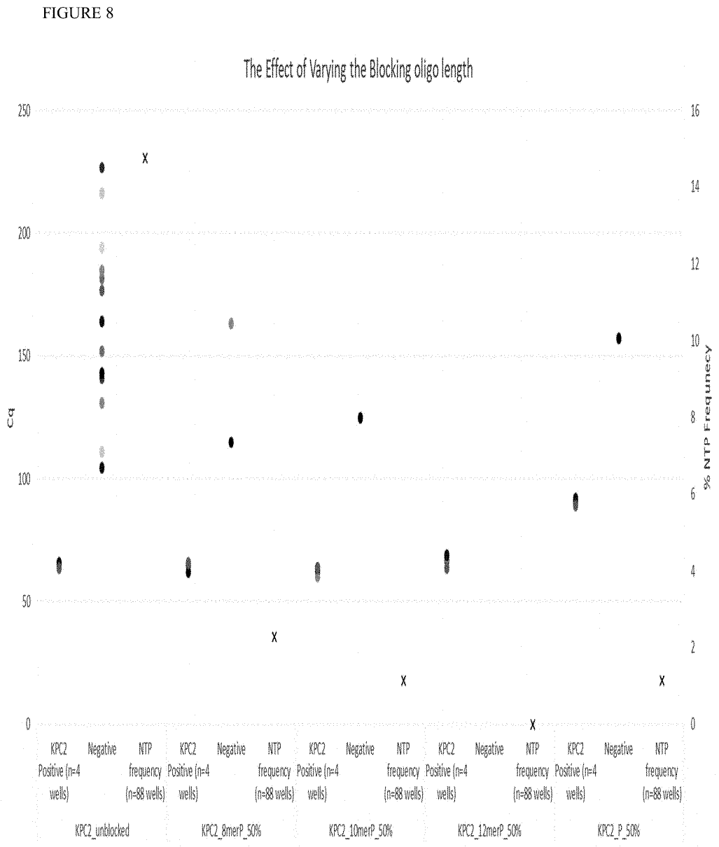

In the experiment shown in FIG. 8. KPC2f is a LAMP primer set that targets the KPC2 gene of Klebsiella and Ecoli. The X axis identifies the Cq of KPC2 Positives true positive LAMP reactions containing 2000 genomes of Klebsiella pseudomonas DNA (KPC2+) as well as NTP Cq and frequency for No Template False Positive wells (NTP) on a 96 well qPCR Plate for each condition. The left Y axis identifies the Cq (quantification cycle, 1 cycle=15 seconds) or speed of the reaction. Each dot represents a single well Cq. The frequency of false positives (No template positives, NTP) is represented with an x symbol and the right Y axis designates % frequency which is the frequency of wells showing NTPs within 300 scans. KPC2f oligos with a 3′ Phosphate blocking group and oligo lengths varying from 8 nt (KPC2_8merP), 10 nt (KPC2_10merP), and 12 nt (KPC2_12merP), were added at 50% the standard LAMP oligo concentration along with 100% standard unblocked oligo where indicated. The Cq for the positive control template positive reactions was around a Cq of 65 for all of the truncated blocked oligos (8mer, 10mer and 12mer). The full length blocked oligos reduced the template positive reaction from a Cq of 65 to 90. The percentage of NTP wells went from 15% in the unblocked control to around 2% for the 8 mer blocked reactions, 1% for the 10mer blocked reactions and there were no NTPs in the 12 mer blocked reactions. The full length blocked reaction had a NTP frequency of around 1%. The Cq differential between TP and NTP increased from a 40 in the unblocked reactions to 50 for the 8mer, 60 for the 10mer, >185 for the 12mer and 67 for the full length blocked reactions.

In the experiment shown in FIG. 9. C2_fs1 is a LAMP primer set that targets the KPC2 gene of Klebsiella and Ecoli. The X axis identifies the Cq of KPC2 Positives true positive LAMP reactions containing 2000 genomes of Klebsiella pseudomonas (KPC2+) as well as NTP Cq and frequency for No Template False Positive wells (NTP) on a 96 well qPCR Plate for each condition. The left Y axis identifies the Cq (quantification cycle, 1 cycle=15 seconds) or speed of the reaction. Each dot represents a single well Cq. The frequency of false positives (No template positives, NTP) is represented with an x symbol and the right Y axis designates % frequency which is the frequency of wells showing NTPs within 200 scans. Full length oligos with either a 3′ Phosphate blocking group (KPC2_P), a 3′ 3 carbon (KPC2_3C3) or an additional 3′ dideoxy C base blocking group (KPC_ddp) were added at 50% the standard LAMP oligo concentration along with 100% standard unblocked oligo where indicated. All the oligos with the different 3′ blocking chemistries were purchased from Integrated DNA Technologies. The addition of blocking oligos that had a 3′phosphate reduced the percentage of NTP wells from 10% to around 1%. The 3′ 3 carbon spacer blocked oligos did not have any NTPs in 88 wells nor did the 3′ dideoxy C base containing oligos.

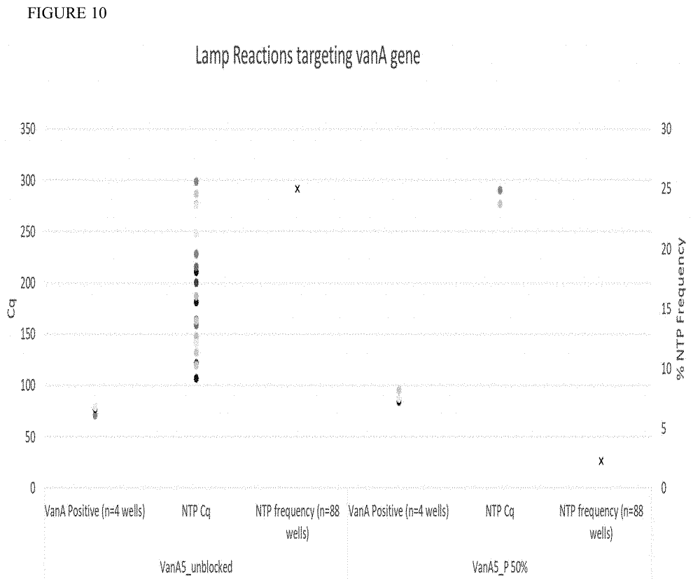

FIG. 10. VanA5 is a LAMP primer set that targets the vanA gene of Enterococcus faecium (vanA+). The X axis identifies the distribution of positives and negatives wells on a 96 well qPCR Plate for each condition of tested while the Y axis identifies the Cq (quantification cycle, 1 cycle=15 seconds) or speed of the reaction. The secondary Y axis identifies the % NTP frequency which is the frequency of wells showing NTPs within 300 scans. At a concentration where the blocked primer (full length primer with a 3′ Phosphate blocking group, VanA5_P) was added at 50% in addition to the 100% standard unblocked oligo concentration , the addition of the blocking primers resulted in a reduction of NTPs from 25% to around 2%. The positive control templated positive reactions were slowed from a Cq of 74 to 87, while the Cq differential between TP and NTP increased from 32 to 190.

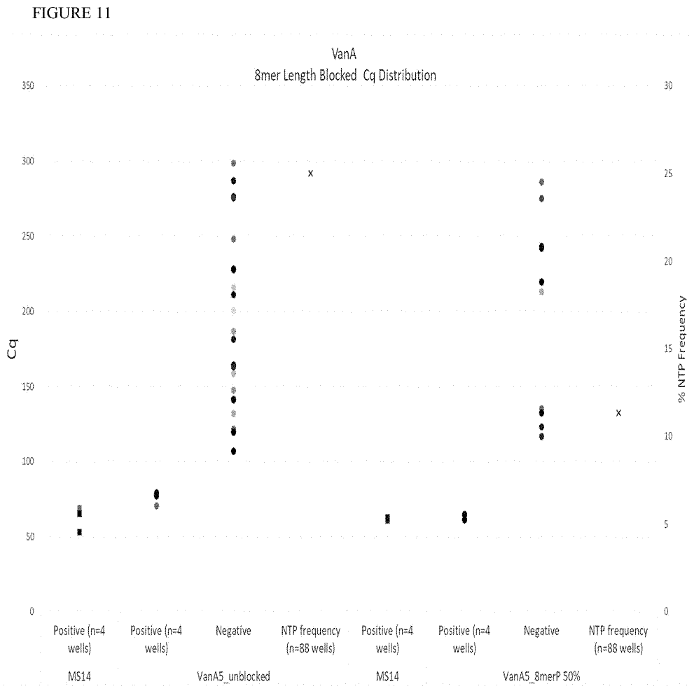

FIG. 11. VanA5 is a LAMP primer set that targets the vanA gene of Enterococcus faecium (vanA+). The X axis identifies the distribution of positives and negatives wells on a 96 well qPCR Plate for each condition of tested while the Y axis identifies the Cq (quantification cycle, 1 cycle=15 seconds) or speed of the reaction. The secondary Y axis identifies the % NTP frequency which is the frequency of wells showing NTPs within 300 scans. At a concentration where the blocking primers (8-mer oligos with a 3′ Phosphate blocking group, VanA5_8mer_P) was added at 50% in addition to the 100% standard unblocked oligo concentration, the addition of the 8-mer blocking primers resulted in a reduction of NTPs from 24% to around 13%. The positive control template positive reactions remained unchanged from a Cq of around 74, while the Cq differential between TP and NTP increased from 32 to 51.

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of” may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed is:

1. A method of detecting or quantifying a target nucleic acid in a nucleic acid sample and reducing the amplification of non-template molecules from the sample, the method comprising
i) incubating a composition comprising a nucleic acid sample comprising a template; one or more first amplification primer set(s); one or more second primer set(s); a polymerase; and deoxynucleotide triphosphates;
ii) amplifying the template; and
iii) detecting or quantifying the amount of amplified template;
wherein the one or more first primer set(s) and the one or more second primer set(s) compete for binding with the template, and the inclusion of one or more second primer set(s) in the composition reduces non-specific amplification products.

2. A method according to claim 1, wherein the amplified template is detected or quantified in real time.

3. A method according to claim 1, wherein the amplification is isothermal.

4. A method according to claim 1, wherein the one or more first primer set(s) is between about 15 and about 50 nucleotides in length.

5. A method according to claim 1, wherein the one or more second primer set(s) is between about 6 and about 50 nucleotides in length.

6. A method according to claim 1, wherein the one or more first primer set(s) is greater in length than the one or more second primer set(s).

7. A method according to claim 1, wherein the at least one second primer set has one or more mismatched nucleotide with the template.

8. A method according to claim 1, wherein the one or more second primer set(s) has two mismatched nucleotides with the template.

9. A method according to claim 1, wherein the at least one second primer set has one or more mismatched nucleotide with the template at the 3′ end of the second primer set.

10. A method according to claim 1, wherein the one or more first primer set(s) have a higher binding affinity for the template in the composition than the one or more second primer set(s).

11. A method according to claim 1, wherein the one or more second primer set(s) comprises modified or non-natural nucleotide analogs.

12. A method according to claim 1, wherein the one or more second primer set(s) has a modified 3′ terminal nucleotide.

13. A method according to claim 12, where the modified 3′ terminal nucleotide of the one or more second primer set(s) is selected from a 3′ phosphate blocking group, a 3′ carbon spacer, or 3′ dideoxy C base blocking group.

14. A method according to claim 10, wherein in the amplification step ii) the modified 3′ terminal nucleotide reduces the amount of amplification of products comprising the one or more second primer sets relative to the amount of amplification of products comprising the one or more first primer sets.

15. A method according to claim 1, wherein the one or more second primer set(s) is between 5% and 90% of the total of first and second primer sets in the composition, between 5% and 50% of the total of first and second primer sets in the composition, between 5% and 40% of the total of first and second primer sets in the composition, between 5% and 30% of the total of first and second primer sets in the composition, between 10% and 80% of the total of first and second primer sets in the composition, between 10% and 50% of the total of first and second primer sets in the composition, between 10% and 40% of the total of first and second primer sets in the composition, between 10% and 30% of the total of first and second primer sets in the composition, between 20% and 70% of the total of first and second primer sets in the composition, between 30% and 60% of the total of first and second primer sets in the composition, or between 40% and 50% of the total of first and second primer sets in the composition.

16. A method according to claim 1, wherein the one or more second primer set(s) is between 5 and 30% of the total percentage by weight of first and second primer sets in the composition.

17. A method according to claim 1, wherein the polymerase is selected from a strand-displacing polymerase and a thermostable polymerase.

18. A method according to claim 1, wherein the reaction mixture is an amplification reaction mixture suitable for amplification by a loop-mediated (LAMP) reaction, stand displacement reaction (SDS), Polymerase Chain Reaction (PCR), a ligase chain reaction (LCR), Isothermal Chimeric Amplification of Nucleic Acids (ICAN), SMart Amplification Process (SMAP), Chimeric Displacement Reaction (RDC), (exponential)-rolling circle amplification (exponential-RCA), Nucleic Acid Sequence Based Amplification (NASBA), Transcription Mediated Amplification (TMA), and Helicase Dependent Amplification (HAD) and Recombinase polymerase amplification (RPA).

19. A method of detecting or quantifying a target nucleic acid in a nucleic acid sample and reducing the amplification of non-template molecules from the sample, the method comprising
i) incubating a composition comprising a nucleic acid sample comprising a template; one or more first amplification primer set(s); one or more second primer set(s); a polymerase; and
deoxynucleotide triphosphates;
ii) amplifying the template; and
iii) detecting or quantifying the amount of amplified template;
wherein the one or more second primer set(s) has a modified 3′ terminal nucleotide selected from a 3′ phosphate blocking group, a 3′ carbon spacer, or 3′ dideoxy C base blocking group,
wherein the one or more first primer set(s) and the one or more second primer set(s) compete for binding with the template, and the inclusion of one or more second primer set(s) in the composition reduces non-specific amplification products.

20. A kit for detecting or quantifying a target nucleic acid in a nucleic acid sample, the kit comprising a i) composition comprising: a) a nucleic acid sample comprising a template; b) one or more first amplification primer sets; c) one or more second primer sets; d) a polymerase; and e) deoxynucleotide triphosphates, wherein during an amplification the one or more first primer set(s) and the one or more second primer set(s) compete for binding with the template, and the inclusion of one or more second primer sets in the composition reduces non-specific amplification products when the template is amplified, and ii) instructions for use of a method according to claim 1.


SEQUENCE LISTING (65 sequences)

SEQ ID NO: 1 - DNA, 18 bp, Artificial Sequence
  Description of Artificial Sequence Synthetic primer
ggcggagttcagctccag

SEQ ID NO: 2 - DNA, 19 bp, Artificial Sequence
  Description of Artificial Sequence Synthetic primer
ccgttacggcaaaaatgcg

SEQ ID NO: 3 - DNA, 39 bp, Artificial Sequence
  Description of Artificial Sequence Synthetic primer
ctgaaggagttgggcggcccgtccagacggaacgtggta

SEQ ID NO: 4 - DNA, 37 bp, Artificial Sequence
  Description of Artificial Sequence Synthetic primer
tgtattgcacggcggccggtggtcacccatctcggaa

SEQ ID NO: 5 - DNA, 18 bp, Artificial Sequence
  Description of Artificial Sequence Synthetic primer
aattggcggcggcgttat

SEQ ID NO: 6 - DNA, 18 bp, Artificial Sequence
  Description of Artificial Sequence Synthetic primer
ggcggagttcagctccag

SEQ ID NO: 7 - DNA, 19 bp, Artificial Sequence
  Description of Artificial Sequence Synthetic primer
ccgttacggcaaaaatgcg

SEQ ID NO: 8 - DNA, 39 bp, Artificial Sequence; identical to SEQ ID NO: 3
  Description of Artificial Sequence Synthetic primer

SEQ ID NO: 9 - DNA, 37 bp, Artificial Sequence; identical to SEQ ID NO: 4
  Description of Artificial Sequence Synthetic primer

SEQ ID NO: 10 - DNA, 18 bp, Artificial Sequence
  Description of Artificial Sequence Synthetic primer
aattggcggcggcgttat

SEQ ID NO: 11 - DNA, 8 bp, Artificial Sequence
  Description of Artificial Sequence Synthetic primer
agctccag

SEQ ID NO: 12 - DNA, 8 bp, Artificial Sequence
  Description of Artificial Sequence Synthetic primer
aaaatgcg

SEQ ID NO: 13 - DNA, 8 bp, Artificial Sequence
  Description of Artificial Sequence Synthetic primer
acgtggta

SEQ ID NO: 14 - DNA, 8 bp, Artificial Sequence
  Description of Artificial Sequence Synthetic primer
tctcggaa

SEQ ID NO: 15 - DNA, 8 bp, Artificial Sequence
  Description of Artificial Sequence Synthetic primer
ggcgttat

SEQ ID NO: 16 - DNA, 10 bp, Artificial Sequence
  Description of Artificial Sequence Synthetic primer
tcagctccag

SEQ ID NO: 17 - DNA, 10 bp, Artificial Sequence
  Description of Artificial Sequence Synthetic primer
caaaaatgcg

SEQ ID NO: 18 - DNA, 10 bp, Artificial Sequence
  Description of Artificial Sequence Synthetic primer
gaacgtggta

SEQ ID NO: 19 - DNA, 10 bp, Artificial Sequence
  Description of Artificial Sequence Synthetic primer
catctcggaa

SEQ ID NO: 20 - DNA, 9 bp, Artificial Sequence
  Description of Artificial Sequence Synthetic primer
cggcgttat

SEQ ID NO: 21 - DNA, 12 bp, Artificial Sequence
  Description of Artificial Sequence Synthetic primer
gttcagctccag

SEQ ID NO: 22 - DNA, 12 bp, Artificial Sequence
  Description of Artificial Sequence Synthetic primer
ggcaaaaatgcg

SEQ ID NO: 23 - DNA, 12 bp, Artificial Sequence
  Description of Artificial Sequence Synthetic primer
cggaacgtggta

SEQ ID NO: 24 - DNA, 12 bp, Artificial Sequence
  Description of Artificial Sequence Synthetic primer
cccatctcggaa

SEQ ID NO: 25 - DNA, 11 bp, Artificial Sequence
  Description of Artificial Sequence Synthetic primer
ggcggcgttat

SEQ ID NO: 26 - DNA, 18 bp, Artificial Sequence
  Description of Artificial Sequence Synthetic primer
ggcggagttcagctccag

SEQ ID NO: 27 - DNA, 19 bp, Artificial Sequence
  Description of Artificial Sequence Synthetic primer
ccgttacggcaaaaatgcg

SEQ ID NO: 28 - DNA, 39 bp, Artificial Sequence; identical to SEQ ID NO: 3
  Description of Artificial Sequence Synthetic primer

SEQ ID NO: 29 - DNA, 37 bp, Artificial Sequence; identical to SEQ ID NO: 4
  Description of Artificial Sequence Synthetic primer

SEQ ID NO: 30 - DNA, 18 bp, Artificial Sequence
  Description of Artificial Sequence Synthetic primer
aattggcggcggcgttat

SEQ ID NO: 31 - DNA, 19 bp, Artificial Sequence
  Description of Artificial Sequence Synthetic primer
ggcggagttcagctccagc

SEQ ID NO: 32 - DNA, 20 bp, Artificial Sequence
  Description of Artificial Sequence Synthetic primer
ccgttacggcaaaaatgcgc

SEQ ID NO: 33 - DNA, 40 bp, Artificial Sequence
  Description of Artificial Sequence Synthetic primer
ctgaaggagttgggcggcccgtccagacggaacgtggtac

SEQ ID NO: 34 - DNA, 38 bp, Artificial Sequence
  Description of Artificial Sequence Synthetic primer
tgtattgcacggcggccggtggtcacccatctcggaac

SEQ ID NO: 35 - DNA, 19 bp, Artificial Sequence
  Description of Artificial Sequence Synthetic primer
aattggcggcggcgttatc

SEQ ID NO: 36 - DNA, 19 bp, Artificial Sequence
  Description of Artificial Sequence Synthetic primer
agcgtcgtttctccctcaa

SEQ ID NO: 37 - DNA, 22 bp, Artificial Sequence
  Description of Artificial Sequence Synthetic primer
tcctccgcttattgatatgctt

SEQ ID NO: 38 - DNA, 41 bp, Artificial Sequence
  Description of Artificial Sequence Synthetic primer
cgccttaccactaccgtctttcaccgctgggtttggtgttg

SEQ ID NO: 39 - DNA, 43 bp, Artificial Sequence
  Description of Artificial Sequence Synthetic primer
taaccaaaaacattgcttgcggcggcgggtagtcctacctgat

SEQ ID NO: 40 - DNA, 25 bp, Artificial Sequence
  Description of Artificial Sequence Synthetic primer
gacctaagccattgtcaaagcgatc

SEQ ID NO: 41 - DNA, 19 bp, Artificial Sequence
  Description of Artificial Sequence Synthetic primer
agcgtcgtttctccctcaa

SEQ ID NO: 42 - DNA, 22 bp, Artificial Sequence
  Description of Artificial Sequence Synthetic primer
tcctccgcttattgatatgctt

SEQ ID NO: 43 - DNA, 41 bp, Artificial Sequence; identical to SEQ ID NO: 38
  Description of Artificial Sequence Synthetic primer

SEQ ID NO: 44 - DNA, 43 bp, Artificial Sequence; identical to SEQ ID NO: 39
  Description of Artificial Sequence Synthetic primer

SEQ ID NO: 45 - DNA, 25 bp, Artificial Sequence
  Description of Artificial Sequence Synthetic primer
gacctaagccattgtcaaagcgatc

SEQ ID NO: 46 - DNA, 8 bp, Artificial Sequence
  Description of Artificial Sequence Synthetic primer
tccctcaa

SEQ ID NO: 47 - DNA, 8 bp, Artificial Sequence
  Description of Artificial Sequence Synthetic primer
atatgctt

SEQ ID NO: 48 - DNA, 8 bp, Artificial Sequence
  Description of Artificial Sequence Synthetic primer
tggtgttg

SEQ ID NO: 49 - DNA, 8 bp, Artificial Sequence
  Description of Artificial Sequence Synthetic primer
tacctgat

SEQ ID NO: 50 - DNA, 8 bp, Artificial Sequence
  Description of Artificial Sequence Synthetic primer
aagcgatc

SEQ ID NO: 51 - DNA, 20 bp, Artificial Sequence
  Description of Artificial Sequence Synthetic primer
cgcaattgaatcggcaagac

SEQ ID NO: 52 - DNA, 18 bp, Artificial Sequence
  Description of Artificial Sequence Synthetic primer
cctcgctcctctgctgaa

SEQ ID NO: 53 - DNA, 42 bp, Artificial Sequence
  Description of Artificial Sequence Synthetic primer
acgcggcactgtttcccaatacaattgagcaggctgtttcgg

SEQ ID NO: 54 - DNA, 40 bp, Artificial Sequence
  Description of Artificial Sequence Synthetic primer
ttcatcaggaagtcgagccggaggtctgcgggaacggtta

SEQ ID NO: 55 - DNA, 22 bp, Artificial Sequence
  Description of Artificial Sequence Synthetic primer
gtactgcagcctgatttggtcc

SEQ ID NO: 56 - DNA, 20 bp, Artificial Sequence
  Description of Artificial Sequence Synthetic primer
cgcaattgaatcggcaagac

SEQ ID NO: 57 - DNA, 18 bp, Artificial Sequence
  Description of Artificial Sequence Synthetic primer
cctcgctcctctgctgaa

SEQ ID NO: 58 - DNA, 42 bp, Artificial Sequence; identical to SEQ ID NO: 53
  Description of Artificial Sequence Synthetic primer

SEQ ID NO: 59 - DNA, 40 bp, Artificial Sequence; identical to SEQ ID NO: 54
  Description of Artificial Sequence Synthetic primer

SEQ ID NO: 60 - DNA, 22 bp, Artificial Sequence
  Description of Artificial Sequence Synthetic primer
gtactgcagcctgatttggtcc

SEQ ID NO: 61 - DNA, 8 bp, Artificial Sequence
  Description of Artificial Sequence Synthetic primer
ggcaagac

SEQ ID NO: 62 - DNA, 8 bp, Artificial Sequence
  Description of Artificial Sequence Synthetic primer
ctgctgaa

SEQ ID NO: 63 - DNA, 8 bp, Artificial Sequence
  Description of Artificial Sequence Synthetic primer
tgtttcgg

SEQ ID NO: 64 - DNA, 8 bp, Artificial Sequence
  Description of Artificial Sequence Synthetic primer
aacggtta

SEQ ID NO: 65 - DNA, 8 bp, Artificial Sequence
  Description of Artificial Sequence Synthetic primer
tttggtcc